(19) United States
(12) Patent Application Publication
(10) Pub. No.: US 2022/0110547 A1
(43) Pub. Date: Apr. 14, 2022

(54) PROVIDING GUIDANCE DURING REST AND RECOVERY

(71) Applicant: Oura Health Oy, Oulu (FI)

(72) Inventors: Hannu Olavi Kinnunen, Oulu (FI); Harri Olavi Laakkonen, Oulu (FI); Kaisa Tarvainen, Oulu (FI); Heli Tuulia Koskimaki, Oulu (FI); Johanna Leena Kyllikki Still, Oulu (FI); Matias Kukka, Oulu (FI)

(21) Appl. No.: 17/500,023

(22) Filed: Oct. 13, 2021

Related U.S. Application Data
(60) Provisional application No. 63/090,931, filed on Oct. 13, 2020.

(51) Int. Cl. A61B 5/11; A61B 5/01; A61B 5/00; A61B 5/024; A61B 5/08; A61B 5/145

(52) U.S. Cl. CPC A61B 5/1118; A61B 5/01; A61B 5/7275; A61B 5/02405; A61B 5/0816; A61B 2560/02; A61B 5/4806; A61B 5/7267; A61B 5/681; A61B 5/6826; A61B 5/14542

(57) ABSTRACT

Methods, systems, and devices for providing guidance during rest and recovery are described. A method may include receiving physiological data associated with a user from a wearable device. The method may include providing, to a user device associated with the user, a first set of physical activity targets and a first set of activity messages based on the received physiological data, where the first set of physical activity targets and the first set of activity messages associated with a first operational mode associated with the user. The method may include identifying a trigger to transition from the first operational mode to a second operational mode associated with the user. The method may include providing, to the user device based on identifying the trigger, a second set of physical activity targets and a second set of activity messages based at least in part on the received physiological data, where the second set of physical activity targets and the second set of activity messages associated with the second operational mode.

### CROSS REFERENCE

The present Application for Patent claims the benefit of U.S. Provisional Patent Application No. 63/090,931 by LAAKKONEN et al., entitled “PROVIDING GUIDANCE DURING REST AND RECOVERY,” filed Oct. 13, 2020, assigned to the assignee hereof, and expressly incorporated by reference herein.

### FIELD OF TECHNOLOGY

The following relates to wearable devices and data processing, including techniques for providing activity guidance in the context of wearable devices.

### BACKGROUND

Some wearable devices may be configured to collect physiological data from users, including heart rate, motion data, temperature data, photoplethysmogram (PPG) data, and the like. In some cases, some wearable devices may provide activity goals and other messaging to a user based on acquired physiological data in order to assist the user with improving their overall health. However, conventional techniques for providing activity goals and other messaging may be inaccurate, and may lead to detrimental health effects in some cases. As such, some conventional techniques for providing activity goals and other messaging may be improved.

### BRIEF DESCRIPTION OF THE DRAWINGS

FIG. 1 illustrates an example of a system that supports techniques for providing guidance during rest and recovery in accordance with aspects of the present disclosure.

FIG. 2 illustrates an example of a system that supports techniques for providing guidance during rest and recovery in accordance with aspects of the present disclosure.

FIG. 3 illustrates an example of a process flow that supports techniques for providing guidance during rest and recovery in accordance with aspects of the present disclosure.

FIG. 4 illustrates an example of a process flow that supports techniques for providing guidance during rest and recovery in accordance with aspects of the present disclosure.

FIG. 5 illustrates an example of a process flow that supports techniques for providing guidance during rest and recovery in accordance with aspects of the present disclosure.

FIGS. 6-11 illustrate examples of graphical user interfaces (GUIs) that support techniques for providing guidance during rest and recovery in accordance with aspects of the present disclosure.

FIG. 12 shows a block diagram of an apparatus that supports techniques for providing guidance during rest and recovery in accordance with aspects of the present disclosure.

FIG. 13 shows a block diagram of a wearable application that supports techniques for providing guidance during rest and recovery in accordance with aspects of the present disclosure.

FIG. 14 shows a diagram of a system including a device that supports techniques for providing guidance during rest and recovery in accordance with aspects of the present disclosure.

FIGS. 15 through 17 show flowcharts illustrating methods that support techniques for providing guidance during rest and recovery in accordance with aspects of the present disclosure.

### DETAILED DESCRIPTION

Some wearable devices may be configured to collect physiological data from users, including heart rate, motion data, temperature data, photoplethysmogram (PPG) data, and the like. In some cases, some wearable devices may provide activity goals and other messaging to a user based on acquired physiological data in order to assist the user with improving their overall health. For example, some wearable devices may provide daily step goals or daily calorie consumption goals based on the user's overall health and fitness goals. However, conventional techniques for providing activity goals and other messaging may be inaccurate, and may lead to detrimental health effects in some cases. For example, in cases where a user is suffering from an illness, or is in a pre-symptomatic stage prior to an illness, exercising in accordance with normal activity goals (e.g., activity goals set while the user is healthy) may actually impair the ability of the user's body to fight the illness, and may therefore actually impair the user's overall health. Similarly, when a user is pregnant, the user's normal “healthy” activity goals and related messaging may not be applicable due to the user's altered condition as a result of the pregnancy.

Accordingly, aspects of the present disclosure are directed to techniques which tailor activity goals, physiological parameter baselines (e.g., expectations for sleep, temperature, heart rate), health-related messaging, and other user guidance provided to a user (e.g., via a graphical user interface (GUI)) based on “operational modes” associated with the user. In particular, aspects of the present disclosure are directed to computing devices/applications (e.g., wearable devices) that measure user physiological parameters, process the measured parameters, and provide outputs to users (e.g., via a GUI). The computing devices/applications may operate in a variety of different operational modes (e.g., normal mode, rest mode, recovery mode, pregnancy mode, vacation mode) that define different device/application functionality. In particular, the various operational modes may be associated with different activity goals, messaging, and the like. As such, techniques described herein may enable wearable devices to tailor health-related guidance to users in accordance with different operational states, where the operational states may be determined based on user inputs, physiological data acquired from the user, or both.

For example, some aspects of the present disclosure describe techniques for providing guidance to a user during rest and recovery (e.g., rest mode, recovery mode). For instance, systems and methods described herein may provide guidance during recovery from a stressful period, acute period of illness, or injury, or during periods of diminished physical capability, such as pregnancy. Applications (e.g., mobile health applications) may provide guidance to their users about healthy habits and behaviors so that users may optimize their performance. In some implementations, devices/applications may provide guidance in a periodic manner (e.g., each morning), in a random manner, or prompted by data driven triggers. The guidance may be provided in a specified context, in a personalized form, and at a time when the user is receptive to the guidance.

During a stressful period of time (e.g., during illness, pregnancy, surgery), and for a time following the stressful period, it may feel inappropriate for a user to receive guidance, targets, and/or charts related to physical activity, consistent sleep timing, and/or healthy eating habits when the focus for the user should be on optimal recovery. During a stressful time, and immediately following, instead of aiming at improved performance, the user may wish to recover and get back to normal physical and mental performance. In some cases, the data collected by wearable devices or mobile health applications may be able to detect the onset of a stressful period (e.g., illness, disease), but it may be more difficult to automatically detect when a user's condition has returned to normal levels.

The devices/systems of the present disclosure may adjust guidance provided to users in accordance with different operational modes, such as during recovery (e.g., recovery mode). In some implementations, the devices/systems may compensate for the conditions in which parameters, such as body temperature, heart rate, heart rate variability (HRV) (and corresponding parameters) return to normal levels earlier than the symptoms disappear. The devices/systems may also compensate to determine when it would be ideal for the human body to return to normal mental or physical levels of strain. For example, the techniques may compensate for a delay in getting back to normal after a period of stress, fever, injury, illness, menstruation, pregnancy, and the like. Additionally, the devices/systems may be able to compensate for injury or other meaningful restrictions following some health-related recommendations. Accordingly, the devices/systems described herein may balance the guidance in a variety of conditions.

In some implementations, an application (e.g., mobile health app) may present daily targets, sleep improvement programs, training programs, and nutritional guidance. The guidance may be based on physiological data acquired via a wearable device, user inputs (e.g., user inputted “tags”), and the like. The targets provided by the application may remain relatively constant from day-to-day, or may vary according to a predefined schedule. For example, easy training days, hard training days, and recovery days may alternate according to a schedule (e.g., designed by a sports coach). In some implementations, an application may alter a training program based on measured parameters (e.g., physiological parameters, detected menstrual cycle parameters, etc.). In some cases, an application may also adjust single day activity targets based on a user's readiness status, which may be calculated based on a previous night's sleep, sleep debt, previous day's activity, resting heart rate, and/or body temperature measurements.

In some implementations, an application may include an operational mode that can be enabled/disabled automatically or manually. The operational mode may be configured such that the application experience changes towards being more fitting to the recovery of the user. The operational mode may also be configured such that the application experience omits some or all other health-related targets, particularly those of physical activity and training targets. When a wearable device measures parameters that indicate added stress or potential illness symptoms, the operational mode may be initiated. In some implementations, the application (e.g., GUI) may ask the user if they wish to start a special operational mode in the application that will help them concentrate on recovery. This operational mode may be referred to as a “rest mode.” In some implementations, the user may activate the rest mode from a menu of the application. This type of activation may be used when the need for the rest mode arises from an injury or pain that is not detected automatically by wearable biosignal measurements.

After rest mode is ended, the application health-related guidance may be gradually adjusted towards normal guidance. In some implementations, the termination of rest mode may be triggered by the user (e.g., manually). In some implementations, the application may prompt the user to end the rest mode. In some implementations, the application may terminate rest mode automatically, or prompt the user automatically (e.g., after a number of body status signals like temperature, heart rate, breathing rate, and the like, have been normalized for a predefined period of time). In some implementations, the adjustment may be done in relation to the length of the stressful period and/or severity of symptoms/signals observed. This period of time may be referred to as a “recovery mode.”

Throughout the stress period and the following gradual return to the normal guidance, observations related to body signals may be interpreted for users mainly in light of recovery (e.g., instead of performance improvements). The selection of health-related content presented to users may be made dependent on the special operational modes described herein (e.g., rest mode and recovery mode). Modifying activity targets (e.g., decreasing targets) and changing messaging (e.g., providing recovery guidance) during rest mode and recovery mode may assist a user in increasing life quality and recovery at a time when activity may not be beneficial to recovery.

In some implementations, automatic triggers for the special mode (e.g., rest mode) may include temperature measurement (e.g., wearable/ring measurement) clearly above a user's long term normative values. For example, if T(i)>(mean of T(i−28:i−1)+0.5 C), where T(i) may be the highest 30-min average temperature over the past night, and T(i−28:i−1) are the corresponding values from past 28 days (4 weeks), which may also be marked as T norm. In some aspects, nocturnal body temperature for a user may be based on continuous temperature measurements.

Another example automatic trigger may include a breathing rate clearly above a user's long term normative value (+1.0 breaths/min or more) or a combination of several factors. An example combination of several factors may be a Readiness Score below a threshold value (e.g., 60). The special operational mode may also be advisable for situations that may include being injured (e.g., so that normal physical activity levels are not possible) or another condition, such as a migraine, back pain, work stress, or a burnout.

During rest mode, it may be particularly fitting to avoid high intensity exercises. Accordingly, during rest mode, some or all physical activity-related targets may be disabled. In some implementations, instead of a minimum target, the nature of the target may be inverted so that the target is set as a maximum target that should not be exceeded. Returning to normal guidance during recovery mode may include adjusting daily activity targets (e.g., calories, active minutes, or steps) by starting from zero, or a lowered target, and ending at normal targets. The adjustment may be based on the amount of time that has elapsed during the rest mode and/or the level of stress/illness. The adjustment may be implemented using a weighted average described herein.

In some implementations, if the user is found to have a fever (e.g., 1.0 C greater than the user's baseline temperature), a system may automatically set or determine that the user needs to have at least a minimum length of rest/recovery (e.g., 2 days of rest mode and 2 days of recovery mode). In some implementations, each extra day (or night) with fever may extend both rest mode and recovery mode so that there may need to be one day (night) without fever in rest mode, and at least as many days in recovery mode as the rest mode has lasted. In some implementations, if temperature rise has been greater than a threshold amount (e.g., 1.0 C), the length of the rest period may be increased by extra time (e.g., 1 extra day for every full 0.5 C). Similarly, threshold values for resting heart rate and/or breathing rate may be set. Z scores may be used in these implementations (e.g., via mean and standard deviation, or mean and mean absolute deviation).

In some implementations, during rest mode, reference values (e.g., baseline or normative values) may not be updated. The benefit of this feature to users may be that sensitivity to future temperature increases is not compromised (e.g., a baseline may not drift upwards during fever days).

In some implementations, some conditions may trigger false alarms for rest. For example, “party nights” (e.g., nights with alcohol intake), specific dates related to human hormones (e.g., high progesterone values during the latter half of a menstrual cycle or during menopause), or other conditions may trigger potential false alarms. In some implementations, the application may include algorithms that differentiate between these “false alarms” and other stressful periods that may require longer rest and recovery periods. Example ways to differentiate may include, but are not limited to questions presented to the user; automatically detecting and excluding periodic temperature increases related to 20-35-day menstrual cycles; and detecting an apparent use of sedatives (e.g., alcohol) from a combination of reduced body movements during the first half of night and increased amount of body movement during the second half of the night, or higher relative increase in heart rate compared to normal than the increase in temperature compared to the user's normal or baseline.

Rest mode and recovery mode may feature a custom set of messages (e.g., daily messages) which are designed to guide the users to shift their focus to recovery. For example, during the rest mode messaging period, the application may highlight metrics that can react to strain, such as resting heart rate, HRV, body temperature, sleep efficiency, and total sleep time. After rest mode has been switched off and the user enters recovery mode, the messaging may gradually start guiding the user back to their normal training routines and targets.

During both rest mode and recovery mode, the measurements upon which the messages are based may be taken over consecutive days. The messages may also emphasize metrics and trends that are the most relevant for the specific user's recovery. In rest mode and recovery mode, instead of providing activity goals and training feedback, activity guidance may encourage the user to focus on rest and recovery, but still break up sedentary time.

Techniques described herein may enable for health-related guidance (e.g., activity targets, expected physiological parameter baselines, sleep targets) provided to a user to be tailored in accordance with one or more “operational modes” associated with the user and/or wearable device.

While much of the present disclosure is described in the context of a “rest mode” and “recovery mode,” this is solely for illustrative purposes, and is not to be regarded as a limitation of the present disclosure. In this regard, techniques described herein may be used to tailor guidance (e.g., activity targets, activity messages) provided to users during any number of operational modes including, but not limited to, a normal mode, a rest mode, a recovery mode, a training mode (e.g., marathon training mode, a football season training mode), an illness mode (e.g., COVID-19 mode, flu mode), a surgery mode (e.g., pre-surgery mode, post-surgery mode), a travel mode (e.g., pre- and post-timezone changes), a vacation mode (e.g., holiday mode), a pregnancy mode, a menstrual cycle mode, a menopause mode, a daylight savings mode, and the like. Moreover, while much of the present disclosure is described in the context of tailoring “physical activity targets,” and “physical activity messages” to users based on activated operational modes, techniques described herein may be used to adjust any health-related targets and messaging provided to users based on activated operational modes. Other health-related guidance which may be tailored based on operational modes may include expectations, targets, and baselines for any physiological parameter (e.g., sleep baseline, temperature baseline, respiratory rate baseline, heart rate baseline), as well as expectations, targets, and baselines for scores (e.g., Activity Score, Sleep Score, Readiness Score) and behavioral characteristics (e.g., movement, activity).

As will be described herein, a system may be configured to transition between operational modes based on manual user inputs received from a user. Additionally, or alternatively, the system may automatically transition between operational modes based on physiological data acquired from the user and/or other data (e.g., upcoming travel plans, expected menstrual cycles, days leading up to daylight savings).

Aspects of the disclosure are initially described in the context of systems supporting physiological data collection from users via wearable devices. Additional aspects of the disclosure are described in the context of example process flows, example GUIs, and the like. Aspects of the disclosure are further illustrated by and described with reference to apparatus diagrams, system diagrams, and flowcharts that relate to techniques for providing health-related guidance during different operational modes (e.g., rest mode, recovery mode).

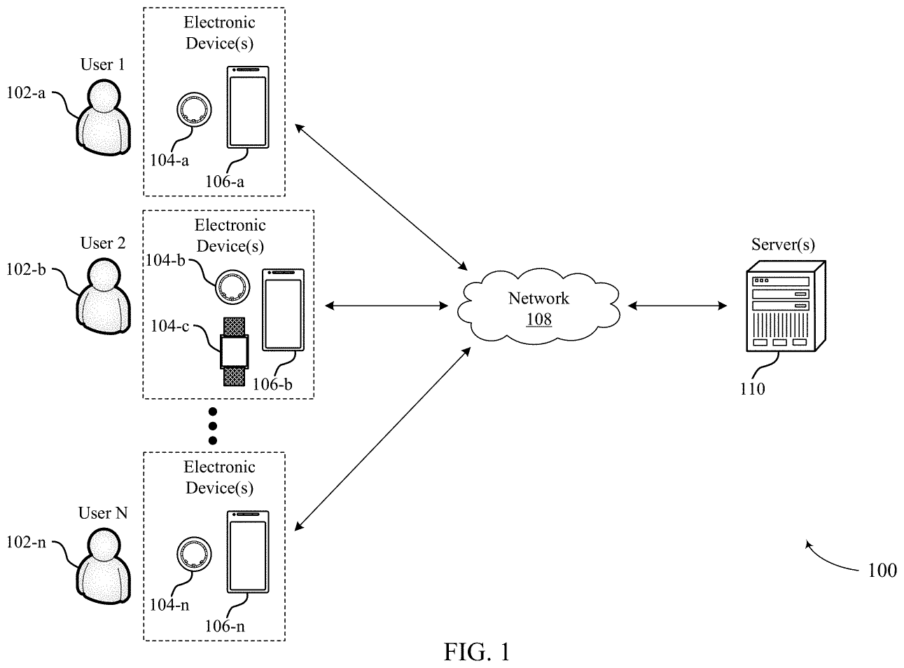

FIG. 1 illustrates an example of a system 100 that supports techniques for providing guidance during various operational modes, in accordance with aspects of the present disclosure. The system 100 includes a plurality of electronic devices (e.g., wearable devices 104, user devices 106) which may be worn and/or operated by one or more users 102. The system 100 further includes a network 108 and one or more servers 110.

The electronic devices may include any electronic devices known in the art, including wearable devices 104 (e.g., ring wearable devices, watch or wrist-worn wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, which may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, HRV, actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) which emit light on the palm-side of a user's finger to collect physiological data based on arterial blood flow within the user's finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices which utilize LEDs which are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time during which a user 102 is asleep, and classify periods of time during which the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time during which the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, which repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models which are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for tailoring health-related guidance to users in accordance with multiple operational modes of the user 102 and/or wearable device 104. For example, the wearable device 104-a associated with the user 102-a may acquire physiological data from the user, including heart rate data, motion data, temperature data, and the like. During a “normal” operational mode, the system may provide health-related guidance to the user based on the user's physiological activity and overall health, including normal physical activity targets (e.g., step goals, calorie goals, standing goals, sleep/rest targets) and normal activity messages (e.g., messages encouraging users to reach their physical activity targets, messages congratulating users for reaching their physical activity targets).

Continuing with the same example, the system 100 may identify a trigger to transition from the normal operational mode to a different operational mode, such as a rest mode. For example, the system 100 may identify that the user is sick or is likely to become sick, and may therefore identify a trigger to transition from the normal operational mode to a rest operational mode. The rest operational mode may be configured to promote rest and recovery for the user 102-a, and may therefore be associated with lowered/reduced physical activity targets and related activity messages (e.g., messages encouraging the user 102-a to rest or take a nap). As such, upon transitioning to the rest mode, the system 100 may tailor physical activity targets and activity messages to help facilitate rest for the user 102-a and allow the user 102-a to prepare for the illness (or upcoming illness).

In some cases, the system 100 may identify the trigger to switch between operational modes (e.g., switch from normal mode to rest mode, and vice versa) based on user inputs received from the user 102-a. For example, the user 102-a may input that they received a positive illness test or that they are beginning to feel ill via the user device 106-a. Additionally, or alternatively, the system 100 may automatically identify triggers for switching between operational modes. For example, the system 100 may recognize that the user is sick (or is likely to become sick) based on physiological data acquired from the wearable device 104-a (e.g., increased temperature, increased respiratory rate, decreased activity). As such, the system 100 may automatically switch between operational modes and/or prompt the user 102-a to confirm or deny a switch between operational modes (e.g., display a message: “It looks like you may be feeling under the weather. Switch to rest mode?”).

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

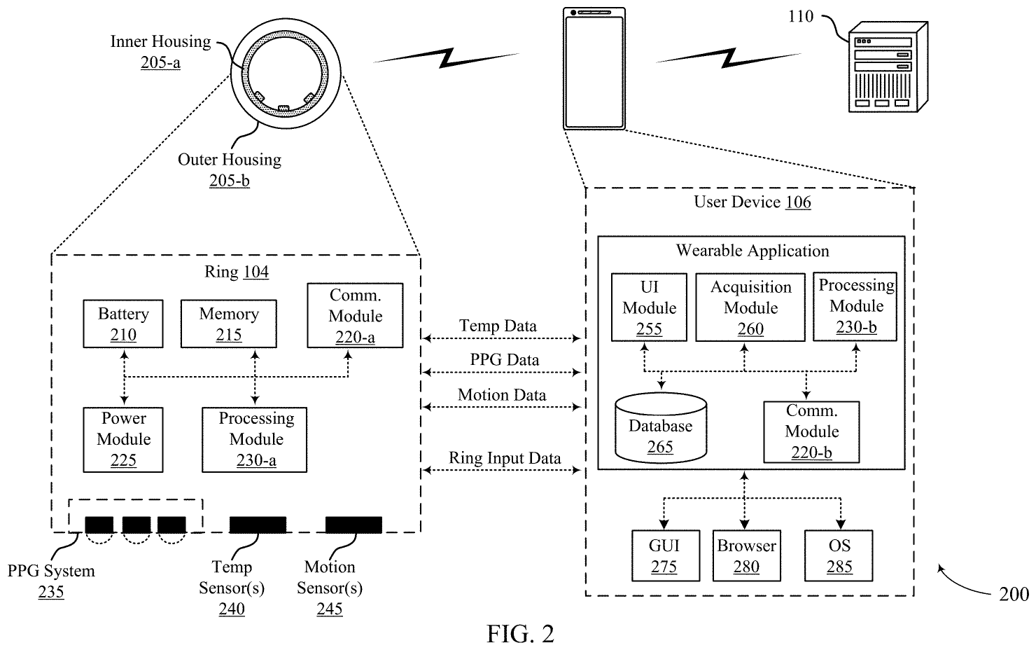

FIG. 2 illustrates an example of a system 200 that supports techniques for providing guidance during various operational modes, in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, which may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components which are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, which may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors which may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user's finger. In some implementations, the PPG system 235 may be configured as a transmissive PPG system 235 in which the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, which may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the 1131s. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) which may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations which require relatively low processing power and/or operations which require a relatively low latency, whereas the user device 106 may transmit the data to the servers 110 for processing operations which require relatively high processing power and/or operations which may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner which is consistent with their sleep schedules. In some cases, users may be able to selectively adjust (e.g., via the GUI) a timing of sleep days relative to calendar days so that the sleep days are aligned with the duration of time in which the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the respective devices of the system 200 may support techniques for tailoring health-related guidance to users in accordance with multiple operational modes of the user and/or wearable device 104. For example, the wearable device 104 may acquire physiological data from a user, including heart rate data, motion data, temperature data, and the like. During a “normal” operational mode, the system may provide health-related guidance to the user based on the user's physiological activity and overall health, including normal physical activity targets (e.g., step goals, calorie goals, standing goals) and normal activity messages (e.g., messages encouraging users to reach their physical activity targets, messages congratulating users for reaching their physical activity targets).

Continuing with the same example, the system 200 may identify a trigger to transition from the normal operational mode to a different operational mode, such as a rest mode. For example, the system 200 may identify that the user is sick or is likely to become sick, and may therefore identify a trigger to transition from the normal operational mode to a rest operational mode. The rest operational mode may be configured to promote rest and recovery for the user, and may therefore be associated with lowered/reduced physical activity targets and related activity messages (e.g., messages encouraging the user to rest or take a nap). As such, upon transitioning to the rest mode, the system 200 may tailor physical activity targets and activity messages to help facilitate the user's rest and allow the user to prepare for the illness (or upcoming illness).

In some cases, the system 200 may identify the trigger to switch between operational modes (e.g., switch from normal mode to rest mode, and vice versa) based on user inputs received from the user. For example, the user may input that they received a positive illness test or that they are beginning to feel ill via the user device 106. Additionally, or alternatively, the system 200 may automatically identify triggers for switching between operational modes. For example, the system 200 may recognize that the user is sick (or is likely to become sick) based on physiological data acquired from the wearable device 104 (e.g., increased temperature, increased respiratory rate, decreased activity). As such, the system 200 may automatically switch between operational modes and/or prompt the user to confirm or deny a switch between operational modes.

For example, in some cases, the system 200 may identify a trigger to switch from a first operational mode (e.g., normal mode) to a second operational mode (e.g., rest mode) based on identifying that one or more physiological parameters satisfy one or more respective thresholds (e.g., based on temperature data exceeding a temperature threshold). Physiological parameters may include, but are not limited to, temperature data, heart rate data, HRV data, respiratory rate data, blood oxygen saturation data, motion data, or any combination thereof.

Similarly, in some aspects, the system 200 may be configured to identify a trigger to switch from one operational mode to another based on calculated health risk metrics. For the purposes of the present disclosure, the term “health risk metric” may be used to refer to any metric or value associated with a relative probability that a user is sick, or is likely to become sick. As such, the term “health risk metric” may be associated with a relative probability that the user will transition from a healthy state to an unhealthy state. The system 200 may be configured to calculate one or more health risk metrics for a user based on acquired physiological data, scores for the user (e.g., Sleep Score, Readiness Score, Activity Score), behavioral data (e.g., sleep timing, sleep duration, sleep quality, activity levels), and the like. In some implementations, the system 200 may be configured to input data (e.g., physiological data, scores) into a classifier (e.g., machine learning classifier, neural network), where the classifier is configured to calculate health risk metrics for the user. In such cases, the system may be configured to identify triggers to switch between operational states based on calculated health risk metrics satisfying (or failing to satisfy) one or more thresholds. Stated differently, the system 200 may identify a potential health risk for the user based on a health risk metric satisfying one or more thresholds, and may thereby identify a trigger to transition between operational states based on the potential health risk.

As described previously herein, the system 200 may support any number of operational modes, where each individual operational mode may be associated with a set of physical activity targets and/or set of activity messages which are tailored to the respective operational mode. Moreover, in some implementations, a single operational mode may include multiple sets of physical activity targets and/or multiple sets of activity messages, where the system 200 may be configured to select between the respective sets of activity targets and/or sets of activity messages based on one or more parameters or characteristics, including acquired physiological data, manual user inputs, and reasons/motivations/causes for the system 200 operating in the respective operational mode. For example, the system 200 may operate in a “rest mode” in cases where a user is suffering from an illness, as well as in cases where the user is recovering from a broken arm. In such cases, the system 200 may utilize different sets of activity targets/activity messages for the user when they are suffering from illness as compared to when the user is recovering from a broken arm (e.g., activity targets may be higher when the user is in rest mode due to the broken arm as compared to activity targets for when the user is in rest mode due to illness). In other words, the system 200 may utilize different subsets of activity targets/activity messages associated with a given operational state based on a “cause,” or motivation, for the user/system 200 operating within the respective operational state.

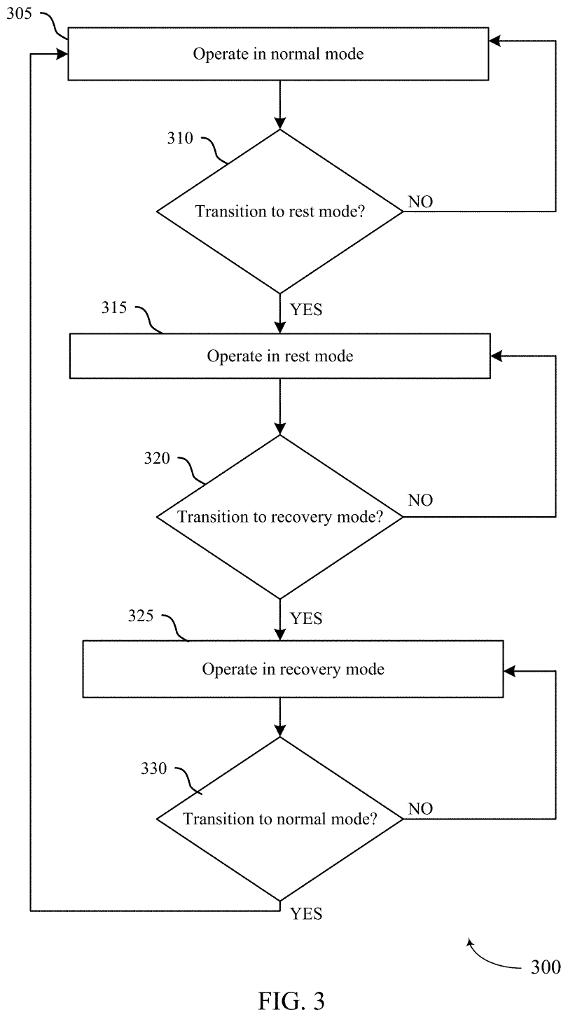

Transitions between operational modes may be further shown and described with reference to FIG. 3.

FIG. 3 illustrates an example of a process flow 300 that supports techniques for providing guidance during various operational modes, in accordance with aspects of the present disclosure. The process flow 300 may implement, or be implemented by, system 100, system 200, or both. In particular, process flow 300 illustrates an example of the system 200 transitioning between operational modes, as described with reference to FIG. 2. In particular, process flow 300 illustrates an example in which the system 200 may transition between a normal mode, a rest mode, and a recovery mode.

At 305, an application (e.g., wearable application 250) may operate in a normal mode according to normal parameters, such as normal activity parameters that promote activity in a healthy individual. For example, while operating in the normal mode (e.g., first operational mode), the system 200 may provide a first set of activity messages and a first set of messages to the user in accordance with the normal mode.

At 310, the application may determine whether to transition from the normal mode to the rest mode. In other words, the application may identify a trigger (or lack thereof) to switch from a first operational mode (e.g., normal mode) to a second operational mode (e.g., rest mode). For example, the application may determine whether to transition to the rest mode based on user input and/or measured physiological parameters that indicate the user has transitioned (or is expected to transition) to an unhealthy state where the user may have added physical stress and/or illness symptoms. In this regard, triggers for transitioning between operational states may be identified based on manual user inputs, automatically identified based on acquired data, or both.

At 315, the application may operate in the rest mode according to rest parameters, such as a reduced/eliminated set of physical activity parameters that are configured to promote recovery of the user. For example, while operating in the rest mode (e.g., second operational mode), the system 200 may provide a second set of activity targets and a second set of activity messages to the user in accordance with the rest mode. In this example, the second set of activity targets may be configured to encourage rest, where the second set of activity messages are configured to promote or encourage the user to meet the second set of activity targets. In some implementations, physical activity targets during the rest mode may be reduced relative to the physical activity targets in the normal mode. Additionally, or alternatively, physical activity targets may be silenced, turned off, or otherwise deactivated during the rest mode to encourage the user to rest.

At 320, the application may determine whether to transition from the rest mode to the recovery mode. In other words, the system 200 may identify a trigger (or lack thereof) to transition from the second operational mode (e.g., rest mode) to a third/intermediate operational mode (e.g., recovery mode). For example, the application may determine whether to transition based on user input and/or measured physiological parameters that indicate the user has reached a threshold level of recovery for a period of time. In this example, the recovery mode may include an intermediate operational mode between the rest mode and the normal mode (e.g., the system 200 transitions from the rest mode to the recovery mode before transitioning from the recovery mode to the normal mode).

In some cases, the system may identify a trigger to transition from the rest mode to the recovery mode based on identifying that a “recovery metric” associated with the user satisfies a threshold recovery level for a period of time. The system 200 may calculate a recovery metric for the user based on acquired physiological data for the user, behavioral data (e.g., sleep timing, sleep duration, activity levels), scores (e.g., Sleep Score, Readiness Score, Activity Score), or any combination thereof.

At 325, the application may operate in the recovery mode according to recovery parameters, such as activity levels that increase from the rest mode recovery levels toward the normal mode activity levels. For example, while operating in the recovery mode (e.g., third/intermediate operational mode), the system 200 may provide a third set of activity targets and a third set of activity messages to the user in accordance with the recovery mode. In this example, the third set of activity targets may be configured to encourage recovery, where the third set of activity messages are configured to promote or encourage the user to meet the third set of activity targets.

At 330, the application may determine whether to transition from the recovery mode to the normal mode. In other words, the system 200 may identify a trigger (or lack thereof) to transition from the third/intermediate operational mode (e.g., recovery mode) back to the first operational mode (e.g., normal mode). For example, the application may determine whether to transition based on user input, a length of the recovery mode, and/or measured physiological parameters that indicate the user has recovered to a sufficiently healthy level.

In some cases, the system may identify a trigger to transition from the recovery mode to the normal mode based on identifying that a “recovery metric” associated with the user satisfies a threshold recovery level for a period of time. Additionally, or alternatively, the system 200 may identify a trigger to transition from the recovery mode to the normal mode based on a duration of time spent in the rest mode, a duration of time spent in the recovery mode, measured physiological parameters (e.g., physiological data collected via the wearable device 104), scores for the user, or any combination thereof. In general, the system 200 may transition from the recovery mode to the normal mode based on identifying that the user has recovered to a sufficiently healthy level (e.g., based on physiological data and/or scores).

As described herein, the system 200 may implement a plurality of different modes. Example additional/alternative modes may include, but are not limited to, a training mode (e.g., marathon training mode, a football season training mode), an illness mode (e.g., COVID-19 mode, flu mode), a surgery mode (e.g., pre-surgery mode, post-surgery mode), a holiday/travel mode, a vacation mode, a pregnancy mode, a menstrual cycle mode, a menopause mode, a daylight savings mode, and the like.

As described herein, the system 200 (e.g., wearable application 250) may provide guidance to a user regarding healthy habits and behaviors so that the user may optimize their performance. During travelling and holidays, there may be a shift in behaviors and biosignals when a person is changing time zones. The guidance during holidays/travel may be periodic (such as every morning), random, or prompted by data driven triggers. The guidance may also be given in the right context and in a personalized form at a time when the user is receptive to the guidance. Although the travel and holiday mode may be described herein as a single holiday mode, in some implementations, holiday mode and travel mode may be separate modes having separate triggers and operations.

During travelling and holidays, it may feel inappropriate to a user to receive guidance, targets, or charts related to consistent sleep timing, healthy eating habits, and/or optimizing readiness. For example, receiving such guidance, targets, or charts may feel inappropriate when experiencing jetlag and living against circadian rhythms. Instead of aiming at improved performance, the user may wish to recover, get back to normal physical and mental performance, and find their optimal moments in the current situation.

Wearable devices 104, or other computing devices (e.g., user device 106, servers 110), may collect data that indicates time zone shift and can help identify periods when modified and personalized guidance may be beneficial. The data collected by devices may be used to identify the onset of travelling when there is a time zone shift. How this shift may affect their biosignals, schedules, and energy may be based on their individual baselines and circadian rhythms. In some cases, body temperature, heart rate, and HRV (and corresponding parameters) may be at different levels due to this shift in schedules. This may also be seen on holidays when people tend to shift their sleep times and their activity periods. Normal messaging may be viewed as negative feedback as compared to a user's expectations during holidays and traveling, and vice versa.

Holiday mode may be described herein relative to other operational modes. In some cases, a general training program (e.g., training mode) may be targeted to motivate a user to move more frequently, longer, and with adequate intensity. A normal sleep program (e.g., sleep scheduling mode) may encourage regular sleep scheduling. A normal readiness program (e.g., readiness mode) may encourage a user to rest when they have been more active or there is a decrease in their quality of sleep/biosignals. When holiday mode is activated, the system 200 may adjust the bedtime guidance and also credit users if they have activity periods at times helping to change their rhythm to the circadian time at the current location.

In some aspects, the system 200 may provide subtle communications/messaging to a user when acquired physiological data for the respective user deviates from the user's normal baselines. Moreover, the system 200 may be configured to interpret observations related to body signals (e.g., acquired physiological data) by taking the circadian effect into account. In some cases, rest mode may be prioritized over holiday mode in cases where conditions indicate a need for rest mode (e.g., a user is becoming over-stressed) during the holiday mode. If rest mode is triggered during holiday mode (e.g., while traveling), the system 200 may begin operating in rest mode instead, such that rest mode is prioritized over other modes.

During holiday mode, sleep timing/consistency and bedtime guidance may be modified to resemble the changed situation. Additionally, the measured biosignals, such as temperature, heart rate, and HRV may be communicated in a manner so that a user can understand that there is a physical strain affecting their body due to travelling and how to adjust their daily schedules. In some implementations, more subtle communication targeting may help the user feel more energetic.

Holiday mode may be activated in a variety of ways. In some implementations, holiday mode may be activated in response to a change in time zones (e.g., a time zone shift greater than 1 hour). In some implementations, holiday mode may be activated in response to a sleep time shift greater than a user's normal weekly variation for two or more consecutive days where, in one example, a shift of at least 0.5 hours above normal weekly variation can be used as a trigger where the algorithm can include exclusion of weekends. In some implementations, holiday mode may be activated in response to activity, such as when more activity (in a sense of time spread and amount) is recognized based on a user's normal routines. In some implementations, holiday mode may be activated in response to feedback from another application, such as a calendar application and/or out-of-office messages. In some implementations, holiday mode may be activated in response to user input (e.g., a manual user input indicating “I'm on holiday.”).

In some aspects, the system 200 may identify a trigger to transition from holiday mode (and/or other operational modes) to normal mode in a variety of scenarios. In other words, the system 200 may be configured to identify any number of triggers for transitioning between operational modes.

For example, the system 200 may transition from holiday mode to normal mode based on a number of days in the new time zone, biosignals (e.g., physiological data), and/or user daily routines. For instance, the system 200 may transition from holiday mode to normal mode when there have been as many days in the new time zone as the difference between time zones, and there is no clear difference in biosignals and/or daily routines compared to baseline. As another example, the system 200 may transition from holiday mode to normal mode when the time zone is shifted back and there has been a long enough adjustment time. In a specific case, the system 200 may take into account the amount of time zones shifted and the duration of travelling and/or the change from the user's normal baselines. For example, three days with time zone shift of +12 may correspond to a length of three days, while twelve days with time zone shift of +12 may correspond to a length of twelve days. This may be gradually shorter or longer based on sleep metrics and/or biosignals compared to the user's baseline. In some implementations, the system 200 may transition from holiday mode to normal mode based on user input. In some cases, the transition may occur when there is no clear difference in biosignals and/or daily routines compared to the user's baseline.

In this regard, holiday mode (and other operational modes) may be enabled and disabled manually, automatically, or both. Holiday mode may change the application experience towards being more fitting for travelling with time zone shift or a period of holiday. In some implementations, holiday mode may omit or communicate some sleep and readiness targets differently, such as sleep consistency and timing and recovery index. For example, in some cases, holiday mode may be initiated when a wearable device 104 and/or user device 106 determines a time zone shift, or identifies additional parameters that indicate a change in daily routines. In some implementations, the system 200 (e.g., wearable application 250) may ask or prompt the user if they wish to start holiday mode, which may then help them to adjust their routines and concentrate on predefined adjusted health and sleep parameters. In some implementations, the user may activate holiday mode from a menu of a mobile health application. This may be useful when the holiday/travel does not include a time zone shift or change in daily routines. For example, the GUI 275 of the user device 106 may display a list of supported operational modes, where the user may be able to select the desired operational mode. In other cases, the user may be able to define and create new operational modes, where the user may be able to customize activity targets and/or activity messages for new operational modes.

After holiday mode has ended, health-related guidance may be gradually adjusted towards normal guidance. In other words, the system 200 may be configured to gradually transition activity targets and/or activity messages when transitioning from one operational mode to another. The termination of an operational mode may be triggered by the user manually. In some implementations, the termination of an operational mode may be selected by a user after being automatically prompted by the application (e.g., in response to a time zone shift back to the user's typical time zone). The adjustment in guidance may be based on the amount of time zones shifted, the duration of travelling, and/or the change from the user's normal baselines. The period during which guidance is adjusted from one operational mode to another may be referred to as an “adjustment mode.” Throughout an operational mode, as well as adjustment mode, observations related to body signals may be interpreted by taking the circadian effect into account, where the system 200 may communicate more subtly to users regarding deviations from their normal baselines.

In some implementations, the system 200 may support other travel-related modes which tailor activity guidance for the user based on various factors, including time zones, latitudes, levels of sunlight, and the like. For example, a user may travel to a different latitude which is in the same time zone as their home, where the new latitude experiences significantly different levels of sunlight as compared to the user's home. In particular, extremely high latitudes may experience very few hours of sunlight per day, which may be a drastic difference between the level of sunlight a user may be accustomed to. Varying levels of sunlight may affect a user's sleep schedules, circadian rhythm, activity, and other behavior. In this regard, the system 200 may identify that a user has traveled to a different latitude, and may trigger a “sunlight exposure mode” or some other latitude or sunlight-related mode. A sunlight exposure mode may tailor guidance that is provided to a user to encourage the user to actively adjust their amount of sun exposure, and may provide other guidance to help the user adjust their activities and sleep schedules in light of the new latitude and/or level of sunlight. Moreover, the system 200 may support additional “intermediary modes” to help the user ease into their normal routines once the user returns home to their normal latitude/level of sunlight.

In some aspects, the system 200 may support a “pregnancy mode” which is configured to tailor health-related guidance provided to pregnant users. A pregnancy mode may modify activity targets and activity-related guidance for pregnant users. For instance, a pregnancy mode may reduce activity intensity expectations, but may increase movement reminders provided to the user, which may better align with physical expectations for a pregnant user. Moreover, a pregnancy mode may adjust other health-related expectations and algorithms used to calculate scores for a user. For example, a pregnancy mode may adjust expectations associated with an amount/type of sleep a pregnant user should get, and adjust expectations associated with other physiological parameters, such as respiration rate, resting heart rate, body temperature, and the like. In this regard, by adjusting expectations associated with physiological parameters for pregnant users, the system 200 may more accurately calculate scores (e.g., Activity Scores, Sleep Scores, Readiness Scores) based on normal, expected physiological responses experienced by pregnant users.

Further, in some aspects, the system 200 may support additional or alternative operational modes associated with a pregnancy mode, including operational modes which help guide a user back to their normal activity levels and physiological parameters following pregnancy. As such, the system 200 may support one or more intermediary modes between a pregnancy mode and a normal mode, including a post-natal recovery mode, a post-natal ramp-up mode, and the like. For example, a post-natal recovery mode may tailor guidance provided to the user which is intended to facilitate rest and recovery in order to help the user recover from pregnancy.

In some implementations, in addition to providing different sets of Activity Scores/activity messages to users based on the corresponding operational states, the system 200 may be configured to calculate scores for the user (e.g., Sleep Score, Readiness Score, Activity Score) differently while operating in accordance with the respective operational states. For example, in some cases, the system 200 may calculate scores (e.g., Sleep Score, Readiness Score, Activity Score) using a first algorithm (or first set of weights) while operating in the first operational state, and may calculate scores using a second algorithm (or second set of weights) while operating in the second operational state. For instance, a first algorithm for score calculation associated with a normal operational mode may result in a decrease in a user's Readiness Score if the user takes a nap late in the evening. Comparatively, a second algorithm for score calculation associated with a rest operational mode may result in an increase in the user's Readiness Score if the user takes a nap in the late evening. This is consistent with the rest mode prioritizing rest for the user.

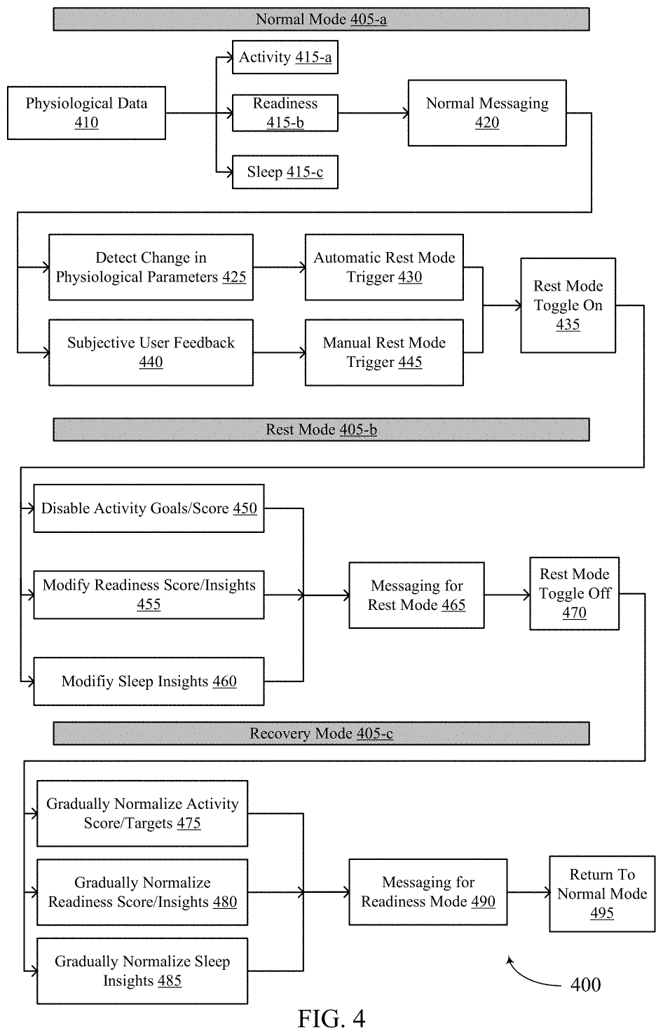

FIG. 4 illustrates an example of a process flow 400 that supports techniques for providing guidance during various operational modes, in accordance with aspects of the present disclosure. The process flow 400 may implement, or be implemented by, system 100, system 200, process flow 300, or any combination thereof. In particular, process flow 400 illustrates an example of the system 200 transitioning between operational modes, as described with reference to FIG. 2. In particular, process flow 400 illustrates an example which describes changes in operation from normal mode to rest mode, and from rest mode to recovery mode in a mobile health application (e.g., wearable application 250).

As shown in FIG. 4, the system 200 may operate in a normal mode 405-a. While in the normal mode 405-a, the system 200 may acquire physiological data 410 via a wearable device 104 (e.g., wearable ring device 104). The system 200 may be configured to calculate various scores for the user based on acquired physiological data 410, including an Activity Score 415-a, a Readiness Score 415-b, and a Sleep Score 415-c. The system 200 may be configured to provide a set of physical activity targets (e.g., calorie targets, step goals) to the user via the user device 106.

Additionally, the system 200 may be configured to provide a set of activity messages to the user, where the activity messages are associated with (e.g., correspond to) the normal mode 405-a. In other words, the system 200 may provide normal messaging 420 to the user, where the normal messaging 420 includes messages that promote the set of activity targets associated with the normal mode 405-a. The normal messaging 420 may include messages associated with the respective scores. For example, a message associated with the user's Activity Score 415-a may include: “Keep yourself active throughout the day, balance between training and recovery days.” By way of another example, a message associated with the user's Readiness Score 415-b may include: “Balance activity and rest, push your boundaries when you're ready,” where a message associated with the user's Sleep Score 415-c may include: “Sufficient and consistent sleep is the key to good readiness.”

Continuing with reference to the process flow 400, the system 200 may detect a change in one or more physiological parameters for the user at 425 (e.g., changes in biosignal data). For example, the system 200 may detect elevated body temperature, or elevated resting heart rate. In such cases, the system 200 may determine an automatic rest mode trigger 430 (e.g., a trigger which is not based on a user input). As such, the system 200 may toggle rest mode on at 435 (e.g., transition from the normal mode 405-a to the rest mode 405-b) in response to the automatic rest mode trigger 430. In additional or alternative cases, the system 200 may receive subjective user feedback 440. For example, a user may input (e.g., via the user device 106) one or more messages or “tags” which indicate that the user may be becoming ill, or may be experiencing some other stressful situation. Manual user inputs may enable the system 200 to identify triggers for switching between operational states in cases where the user feels sick (or is experiencing some other episode), but where acquired physiological data has not changed significantly. In such cases, the system 200 may identify a manual rest mode trigger 445 (e.g., a trigger based on a manual user input). As such, the system 200 may toggle rest mode on at 435 (e.g., transition from the normal mode 405-a to the rest mode 405-b) in response to the manual rest mode trigger 445.

Upon activating the rest mode 405-b, the system 200 may adjust activity targets and/or activity messaging which may be provided to the user. Additionally, or alternatively, the system 200 may adjust how it calculates scores for the user (e.g., switch to a different algorithm for calculating Sleep Scores, Readiness Scores, Activity Scores, etc.). For example, the system 200 may modify and/or disable activity goals, contributors, and/or Activity Score calculation at 450. Similarly, the system 200 may modify Readiness Score contributors and insights at 455, and may modify sleep insights at 460. Subsequently, the system 200 may provide messaging to the user at 465, where the messaging (e.g., activity messages) are associated with the rest mode 405-b. That is, the system 200 may provide messaging which promotes rest based on performing the actions at 450, 455, and 460.

The messaging for rest mode 465 may include messages intended to promote rest during the rest mode 405-b, and may be based on the user's respective scores. For example, a message associated with the user's Activity Score while in the rest mode 405-b may include: “Concentrate on rest.” By way of another example, a message associated with the user's Readiness Score while in rest mode 405-b may include: “Concentrate on rest and recovery to achieve your peak readiness,” where a message associated with the user's Sleep Score while in the rest mode 405-b may include: “All rest is good rest.”

Subsequently, the system 200 may toggle rest mode off at 470. In other words, the system 200 may identify a trigger to transition from the rest mode 405-b to another operational mode (e.g., normal mode 405-a, recovery mode 405-c). As described previously herein, the system 200 may toggle rest mode 405-c off at 470 based on identifying a trigger, where the trigger may be based on a user input (e.g., manual user input) and/or automatically identified based on received physiological data and/or calculated scores.

Upon activating the recovery mode 405-c, the system 200 may adjust activity targets and/or activity messaging which may be provided to the user. Additionally, or alternatively, the system 200 may adjust how it calculates scores for the user (e.g., switch to a different algorithm for calculating Sleep Scores, Readiness Scores, Activity Scores, etc.). For example, the system 200 may gradually normalize Activity Score (e.g., Activity Score calculation), activity goals, and/or activity contributors at 475. Similarly, the system 200 may gradually normalize Readiness Score (e.g., Readiness Score calculation), readiness contributors, and readiness insights at 480, and may gradually normalize sleep insights at 485.

The system 200 may be configured to provide messaging to the user at 490, where the messaging (e.g., activity messages) are associated with the recovery mode 405-c. That is, the system 200 may provide messaging which promotes rest and recovery based on performing the actions at 475, 480, and 485. For example, a message associated with the user's Activity Score while in the recovery mode 405-c may include: “Start easy.” By way of another example, a message associated with the user's Readiness Score while in recovery mode 405-c may include: “Keep taking it easy, but you can start with light activities,” where a message associated with the user's Sleep Score while in the recovery mode 405-c may include: “Keep paying attention to rest and sleep.”

Subsequently, the system 200 may toggle readiness mode 405-c off and return to normal mode 405-a at 495. In other words, the system 200 may identify a trigger to transition from the recovery mode 405-c to another operational mode (e.g., normal mode 405-a). As described previously herein, the system 200 may toggle recovery mode 405-c off and switch to normal mode 405-a at 495 based on identifying a trigger, where the trigger may be based on a user input (e.g., manual user input) and/or automatically identified based on received physiological data and/or calculated scores.

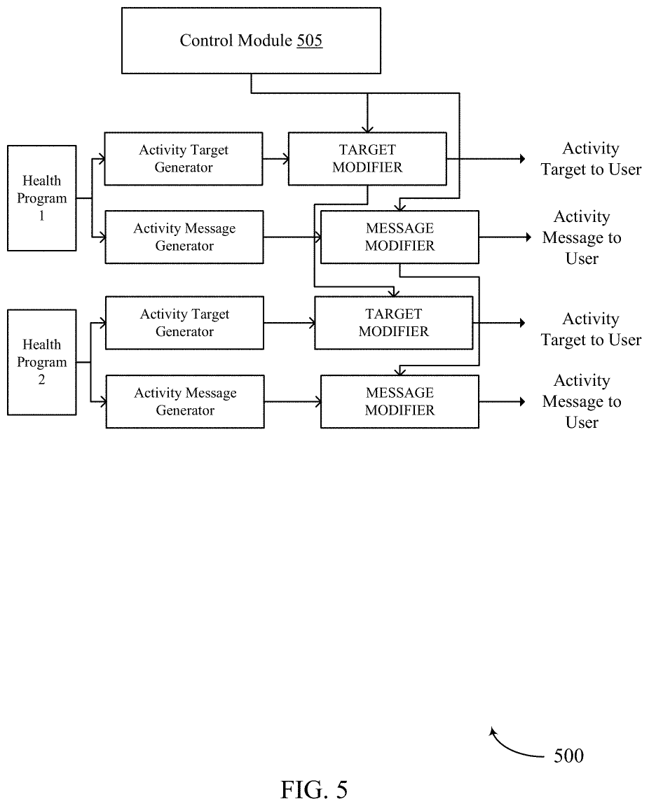

FIG. 5 illustrates an example of a process flow 500 that supports techniques for providing guidance during various operational modes, in accordance with aspects of the present disclosure. The process flow 500 may implement, or be implemented by, system 100, system 200, process flow 300, process flow 400, or any combination thereof. Process flow 500 illustrates an example control diagram for two or more health programs (e.g., one or more operational modes) in a user device 106.

For the purposes of the present disclosure, the term “health program” may be used to refer to a longer-term health/fitness program associated with a user. Example health programs may include but are not limited to: an exercise training program, a sleep program, and a nutrition program, and the like. Each program may also include additional programs (e.g., sub-category programs). For example, training programs may include a muscle training program, a general endurance training program, and/or a health-enhancing training program. Health programs may be used to determine activity targets and related activity messaging for the user. In some implementations, operational modes may be used to selectively modify activity targets/messaging within each respective health program. That is, a user may be actively engaged in a muscle training program, and the system 200 may selectively modify activity targets/messaging provided to the user throughout the muscle training program as the system 200 transitions between different operational modes (e.g., normal mode, rest mode, recovery mode, base level mode, easy mode, intensive mode) throughout the duration of the muscle training program.

The process flow 500 illustrates a control module 505 which may be implemented via one or more components of the system 200 (e.g., wearable device 104, user device 106, servers 110). The control module 505 may include or support multiple operational modes, such as a normal mode, a rest mode, and a recovery mode. The normal, rest, and recovery modes are only example modes. As such, other implementations may include different and/or additional operational modes. For example, other implementations may include four operational modes: a healthy mode, an acute health condition mode, a recovery from an acute health condition mode, and a chronic health condition mode. The two or more programs/operational modes may be centrally controlled by the central controlling block (e.g., control module 505). The control module 505 (e.g., with three operational modes) may set rules for the respective operational modes/health programs. The effect of the prevailing operational mode (e.g., rest mode, recovery mode, normal mode) may be visible in several different health programs/operational modes simultaneously (e.g., by modifying activity targets and activity messages given to users).

For example, as shown in the process flow 500, a first health program may be associated with a first set of activity targets and a first set of activity messages, where a second health program may be associated with a second set of activity targets and a second set of activity messages. In such cases, upon identifying a transition between operational modes, the system 200 (e.g., control module 505) may selectively modify the respective activity targets and/or activity messages of the respective health programs based on the active operational state. The modified activity targets and/or modified activity messages for the respective health program(s) may then be provided to the user (e.g., via the user device 106).

The system 200 may support a general training program that is targeted to motivate a user to move more frequently, longer, and with adequate intensity. A sleep program may encourage regular sleep scheduling and avoiding long naps. In some implementations, when the rest mode is activated for a respective health program, the control module 505 may set a rule for maximum training amounts or intensity for the health program. Additionally, the control module 505 may also credit the user for actions (e.g., other than training) that enhance recovery, such as taking naps. In a specific example, the system 200 may multiply the numeric daily activity target to be 50-100% of the normal, and increase recommendation for relaxing activities by 100%. The control module 505 may be configured to select and/or modify different message types and control the presentation of messages for different activities within a respective health program. For example, the control module 505 may not show a message about negative long-term effects of naps, but instead may show a message about their immediate positive effects. Accordingly, in some implementations, the control module 505 may increase the targeted amount of sleep and increase the priority/occurrence rate of positive messages related to sleep and recovery contributions when operating in the rest or recovery modes (e.g., regardless of the activated health program).

In some implementations, the rest mode may be activated based on measured signals, such as elevated temperature, elevated breathing rate, resting heart rate, decreased HRV, or the like. In some implementations, the rest mode may be activated based on an indication of health risk, such as an illness indication (e.g., a COVID-19 indication) that may be automatically detected or reported by the user. In some implementations, the rest mode may be activated in response to user input, such as user input that indicates an injury (e.g., a broken bone) or an illness (e.g., the flu). In some implementations, rest mode may enable a follow up of symptoms (e.g., using specific tags).

Rest mode may transition to recovery mode. During recovery mode, the rules/settings may gradually be returned to normal mode. For example, the rules may change by x %/day until the normal level is reached (e.g., until the difference <10%).

Rest mode may transition to recovery mode in a variety of ways. For example, rest mode may transition to recovery mode in response to a default time, such as a default elapsed time (e.g., 1 week) and/or a specified future date. As another example, rest mode may transition to recovery mode when measured parameters have returned to normal. In some cases, the control module 505 may add on a set period of time (e.g., 1 week) after measured parameters have returned to normal. As another example, rest mode may transition to recovery mode in response to user input, such as a manual input that indicates the user's health has normalized or that a risk has passed. As another example, rest mode may transition to recovery mode when a health alert/risk indicator has disappeared. In some cases, the control block may add on a set period of time (e.g., 3 days) after the health alert/risk indicator has disappeared.

The length of recovery mode may be calculated based on a variety of factors. In some implementations, the length of the recovery mode may be based on the length of rest mode. For example, the length of the recovery mode may be set to a multiple of the length of the rest mode (e.g., 1-3 times the rest mode). In this example, the time multiplier may be age-dependent, where older people may have a larger multiplier (e.g., an increased recovery time). For example, a 20-year-old user may have recovery time=rest time. As another example, a 40-year-old user may have recovery time=1.5×rest time. As another example, a 60-year-old user may have recovery time=2×rest time. In some implementations, the time multiplier may be based on measured physiological values (e.g., temperature, heart rate, HRV, respiratory rate, etc.). For example, the time multiplier may be based on a maximum temperature measured during the rest mode. In a specific example, if the user's temperature increased by more than 2.0° C., recovery time may equal 2× rest time, otherwise 1×rest time, or gradually longer as a function of the maximum temperature increase observed.

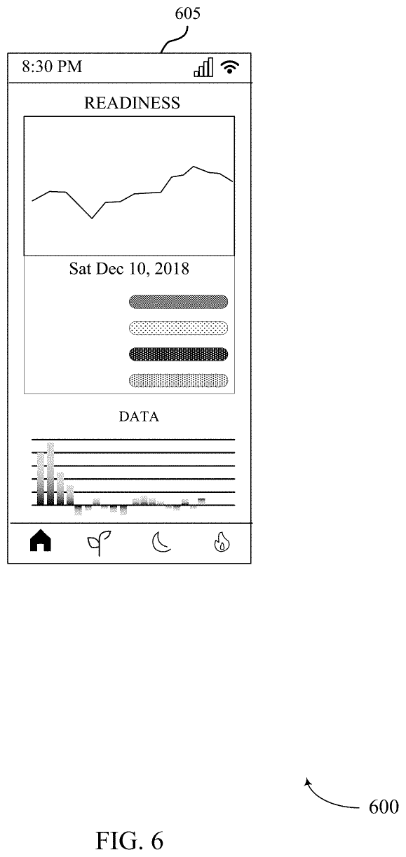

FIGS. 6-11 illustrate examples of GUIs 600-1100 that support techniques for providing guidance during various operational modes, in accordance with aspects of the present disclosure. The GUIs 600-1100 may implement, or be implemented by, aspects of the system 100, system 200, process flow 300, process flow 400, process flow 500, or any combination thereof. For example, the GUIs 600-1100 may include example of the GUI 275 included within the user device 106 illustrated in FIG. 2.

The GUIs 600-1100 illustrate application pages which may be displayed to a user, such as via a GUI 275 of the user device 106 shown and described in FIG. 2. For example, GUI 600 illustrates an application page 605 which displays measured biosignal data (e.g., acquired physiological data), which may be used to trigger a switch between health programs and/or operational states. In some aspects, as shown in the application page 605, the system 200 may be configured to determine normal or baseline levels of physiological parameters for the user (e.g., baseline physiological data). In such cases, the system 200 may be configured to identify significant deviations from the normal/baseline levels in order as triggers to switch between operational modes of the system 200, as described herein.

The application page 605 illustrates how physiological data collected via a wearable device may indicate an onset of a specific acute stressful situation, such as an illness. The acquired physiological data may be used for triggering the rest mode (or acute stressful condition mode). Example triggers may include, but are not limited to: 1) body temperature triggers (e.g., a body temperature deviation greater than 0.5 C from a user's norm), 2) a respiration rate trigger (e.g., respiration rate increased by more than 1 breath per minute), and 3) a resting HR trigger (e.g., resting heart rate increased by more than 10 beats per minute). Different parameters and time windows may be used, such as longer time windows (e.g., longer than one day/night). In some implementations, the system 200 may statistically analyze a user's historical data to determine a normal range (such as median and standard deviation). In these implementations, if several values, or their weighted sum, fall outside the normal range (e.g., by a threshold value), the combination may trigger the rest mode.

In some cases, the system 200 may analyze a user's historical data to determine how the user recovered from the same or similar illness, injury, or other condition in the past (e.g., how long the user took to recover, how the user's physiological data reacted at different stages of the recovery). This analysis may be used to determine durations of time for respective operational modes (e.g., rest mode, recovery mode), to determine triggers for transitioning between operational modes, and the like. Additionally, or alternatively, the system 200 may compare the user's physiological data to that of other user's who have suffered from similar illness, injury, or other condition. By comparing to physiological data of other users, the system 200 may be able to more accurately estimate durations of time for respective operational modes (e.g., rest mode, recovery mode) and determine triggers for transitioning between operational modes.

Another example trigger for specific central block modes, such as the rest mode, may come from a symptom-based risk profiling from a mobile health application or a survey. In some cases, the risk profiling may include wearable device data. In a specific example, the risk profile may have been learned and detected for specific illnesses, such as bacterial infections, viral infections (e.g., influenza AB or COVID-19), or other health conditions.

In some implementations, triggering the rest mode may be based on the time of year. For example, specific times of year may be pre-defined as seasonally higher risk time windows. In this example, the activation of the rest mode may be initiated between a time window (e.g., October to March), or the sensitivity may be higher during that time. In a specific example, a “risk time window” may be set, and if the user has illness symptoms (e.g., elevated breathing) or a diagnosis, the rest mode may be triggered. In this specific example, the risk time window may be seasonal (e.g., during October), and if the user input or measurement of his/her biosignals shows that he/she has an illness (e.g., illness indicating biosignals), the rest mode may be set immediately for a one-week period.

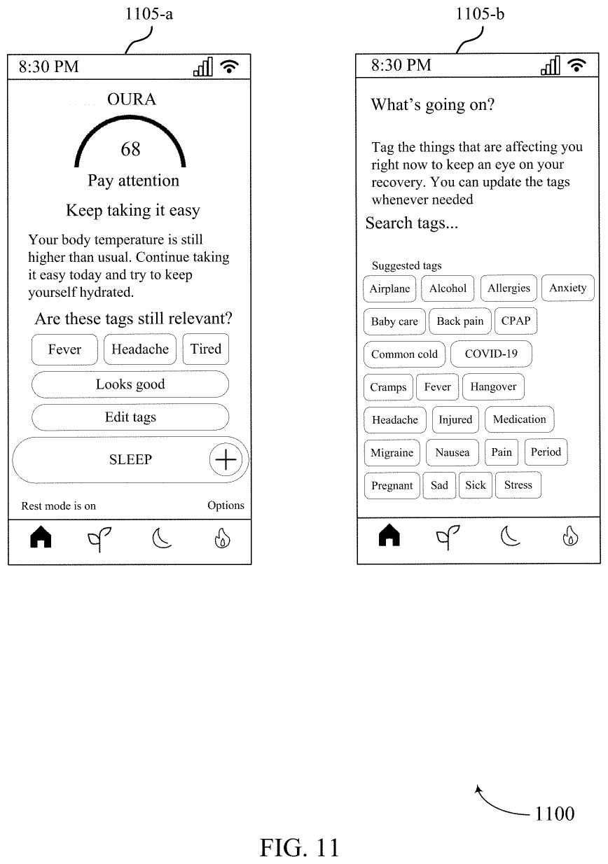

In some implementations, manually inputted tags may be configured to trigger rest mode. For example, user selection of one or more specific tags may trigger rest mode. Tags which may be inputted or selected by users will be further shown and described with reference to FIG. 11.

In some aspects, physical activity targets may be modified from one operational mode to another (e.g., modified during rest mode). For example, in some implementations, no physical training targets may be implemented during rest mode. In some implementations, light intensity activity, such as breaking up sedentary activities, may be implemented during rest mode. In some implementations, sleeping/resting may be emphasized during rest mode (e.g., instead of physical activity).

Messages (e.g., in the GUI 275) may be modified during rest mode. In some implementations, rest mode may include custom messages. For example, rest mode may feature a custom set of daily messages that are designed to guide the users to shift their focus to recovery. During the rest mode messaging period, the application may highlight metrics than can react to strain, such as resting heart rate, HRV, body temperature, sleep efficiency, and total sleep time.

During both rest and recovery mode, the measurements on which the messages are based on may be acquired over consecutive days. Additionally, the messages may emphasize metrics and trends that are the most relevant for that user during recovery. In rest and recovery mode, instead of providing activity goals and training feedback, activity guidance may encourage the user to focus on rest and recovery, but still break up sedentary time.

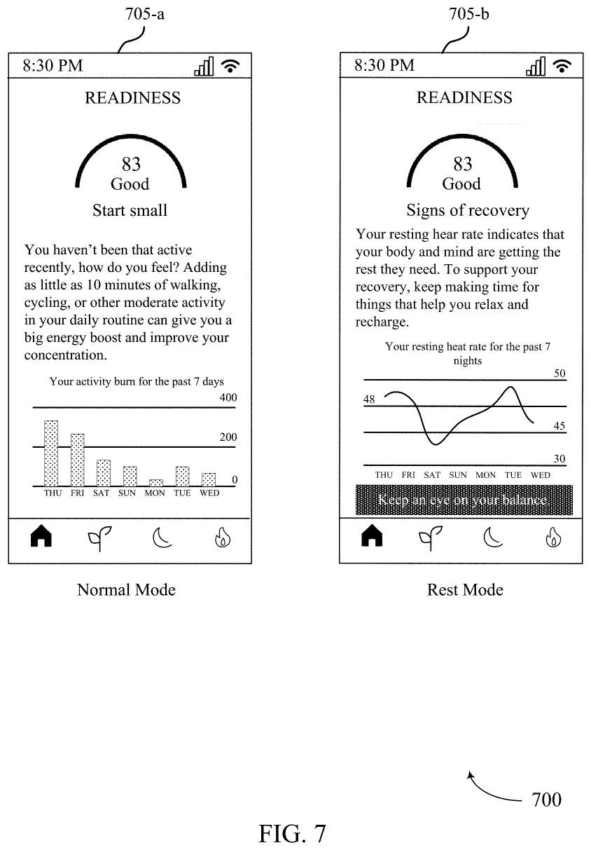

Referring now to FIG. 7, GUI 700 illustrates a set of application pages 705-a, 705-b which may be displayed to a user via GUI 275 of the user device 106 illustrated in FIG. 2. The first application page 705-a illustrates activity targets and activity messaging associated with a normal mode (e.g., normal operational mode). As shown in the first application page 705-a, the system 200 may prompt the user to increase activity levels after a longer period of inactive time during normal mode. However, such a message may not be optimal if the user has suffered from an illness recently. Accordingly, application page 705-b illustrates activity targets and activity messaging associated with a rest mode (e.g., rest operational mode). In particular, the second application page 705-b illustrates an example of an activity message which may be displayed to the same user on the same day (e.g., same physiological data) when rest mode is enabled. As may be seen by comparing the first application page 705-a (normal mode) with the second application page 705-b (rest mode), the guidance provided during rest mode may emphasize recovery metrics and focus on rest instead of prompting the user to become more active.

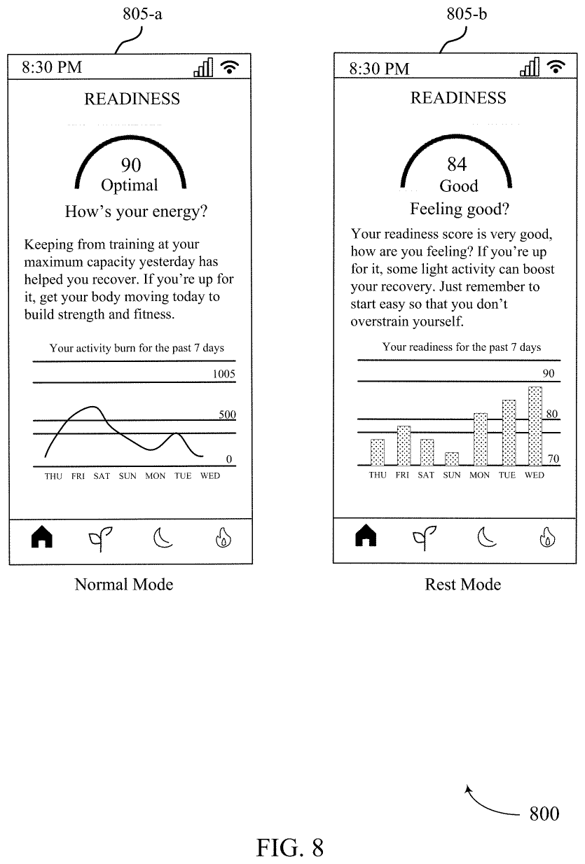

Referring now to FIG. 8, GUI 800 illustrates a set of application pages 805-a, 805-b which may be displayed to a user via GUI 275 of the user device 106 illustrated in FIG. 2. The application pages 805-a and 805-b may illustrate example guidance (e.g., activity targets, activity messages) which may be provided to a user in normal mode and rest mode, respectively. In particular, the application pages 805-a, 805-b may be displayed to the same user in response to the same physiological data, where the only difference is the operational mode in which the system 200 is operating. Referring to GUI 800, it may be assumed that the user has had low activity levels the day before because of feeling unwell. The rest mode may be activated and a message suggesting light activity may be presented to the user after the user has reported (or it has been detected that the user was) feeling unwell the day before. Comparing the first application page 805-a (normal mode) and the second application mode 805-b (rest mode) illustrates a difference in the health application experience when the user has felt unwell the day before, and how the user experience is changed after the rest mode has been enabled.

Additionally, in some implementations, calculation of Readiness Scores may also take into account the enabled operational mode such that the system 200 gives different weights to the parameters that better indicate body status. In other words, the system 200 may calculate readiness/sleep/activity scores differently (e.g., using different algorithms, using different weights) based on which operational state is enabled. For example, with respect to sleep health programs during rest mode, in some implementations, the role of naps may be positive in rest mode, and may be interpreted and communicated as contributing to improved recovery. In normal mode, naps may not be typically recommended, as they can spoil normal circadian rhythms. In other words, a system 200 may calculate sleep and Readiness Scores differently in normal mode and rest mode such that a nap will have a different effect on the user's sleep and Readiness Scores while in normal mode as compared to rest mode.

During the recovery mode, activity targets may be modified. In some implementations, physical training targets may still be reduced. One example of returning to normal guidance during recovery mode is that daily activity targets (such as calories, active minutes, or steps) are adjusted starting from zero, or a very low target, and ending at normal targets after a period of time. In some implementations, the period of time may be as long as the stressful/illness period. For example, the adjustment can be implemented using weighted averages as follows:

normal_target_weight=min((recovery_period_days_so_far+1)/(rest_mode_period_length_days+1),1)

low_target_weight=1−normal_target_weight

activity_target=(normal_target*normal_target_weight)+(low_target*low_target_weight)

If the lowered target is determined dynamically, for example using the biosignals and user's recent history, it may be useful to ensure that the target remains within reasonable limits during the recovery period. For example, soft limits may be used to keep the lowered target between 10% (0.1) and 85% (0.85) of normal target according to the following:

if target_level > 0.8
target_level = 0.8 + (target_level − 0.8) / 4;
endif
if target_level < 0.2
target_level = 0.2 − (0.2 - target_level) / 2;
endif

The following describes some example modifications for targets in a sleep program. In one example, getting more sleep may still be emphasized as it is during the rest mode. However, naps may no longer be recommended unless a person sleeps less than 6.5 hours during the previous night. In normal mode, naps may not be recommended unless a person sleeps less than 5 hours, as an example.

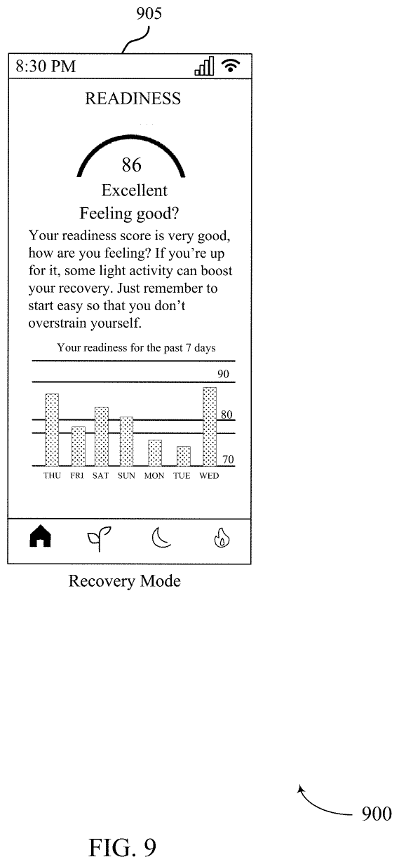

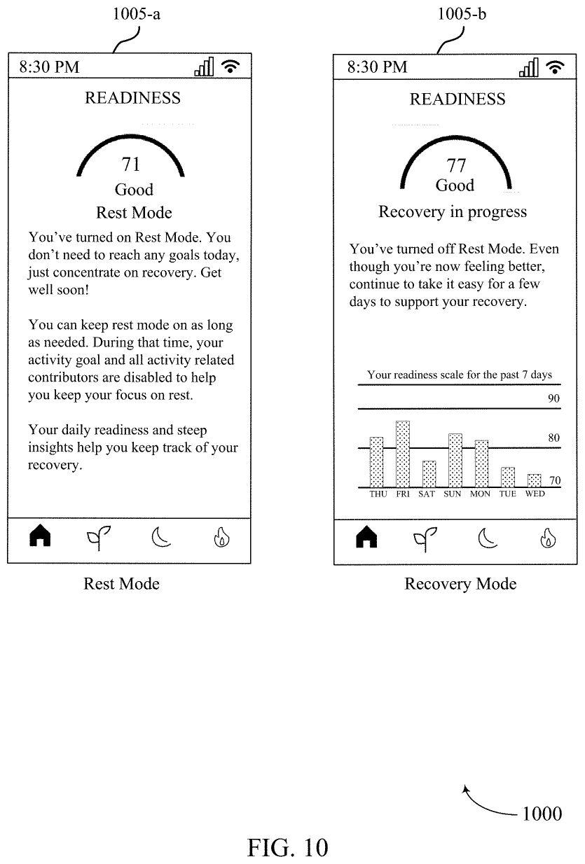

Moreover, activity messages may be modified from one operational mode to another (e.g., modified during recovery mode). For example, after rest mode has been switched off (automatically or manually) and the user enters recovery mode, the messaging may gradually start guiding the user back to their normal training routines and targets. For example, referring now to FIGS. 9 and 10, GUIs 900 and 1000 illustrate application pages 905, 1005-a, and 1005-b, which may be displayed to a user via GUI 275 of the user device 106 illustrated in FIG. 2.

Application page 905 illustrated in FIG. 9 shows an example where rest mode has been disabled and recovery mode has been switched on. As shown in application page 905, the system 200 may display messages which promote recovery while in recovery mode.

Application page 1005-a and application page 1005-b illustrate examples of guidance which may be displayed to a user while in rest mode and recovery mode, respectively. As may be seen by comparing application pages 1005-a and 1005-b, the guidance provided to the user within rest mode and recovery mode may be different, even if the underlying scores and physiological parameters are the same.

Referring now to FIG. 11, GUI 1100 illustrates a set of application pages 1105-a, 1005-b which may be displayed to a user via GUI 275 of the user device 106 illustrated in FIG. 2. In particular, application pages 1005-a and 1005-b illustrate example “tags” that a user may input via a user device 106. The respective “tags” may include subjective and/or objective descriptions of the user's emotions, activities, and/or physical state. The application pages 1105 may illustrate how different operational modes may be used to change how a tagging feature is used. For example, in some cases, the system 200 may emphasize or otherwise encourage users to utilize tags more in the rest mode and recovery mode as compared to the normal mode. Moreover, the application pages 1105 show how suggested tags can be presented to the user.

As noted previously herein, the system 200 may utilize tags inputted/selected by a user to identify triggers for switching between operational states. For example, if a user selects a “pregnancy” tag, the system 200 may switch to a pregnancy operational state, where the activity targets and activity messaging provided to the user are configured to promote healthy pregnancy activity. In this example, the activity targets and activity messaging may change throughout the pregnancy as the user progresses throughout her pregnancy. In some cases, users may be able to select from a set of pre-configured tags. In other cases, a user may be able to input custom tags or insights. Tags may be related to nutrition, caffeine, lifestyle, sports activities, health, and the like.

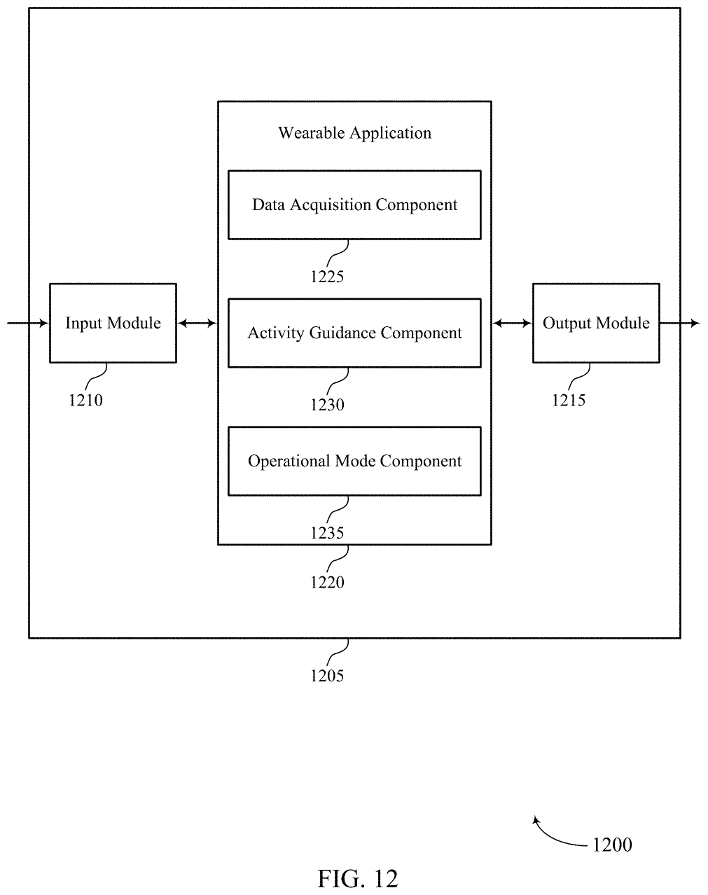

FIG. 12 shows a block diagram 1200 of a device 1205 that supports techniques for providing guidance during rest and recovery in accordance with aspects of the present disclosure. The device 1205 may include an input module 1210, an output module 1215, and a wearable application 1220. The device 1205 may also include a processor. Each of these components may be in communication with one another (e.g., via one or more buses).

The input module 1210 may provide a means for receiving information such as packets, user data, control information, or any combination thereof associated with various information channels (e.g., control channels, data channels, information channels related to illness detection techniques). Information may be passed on to other components of the device 1205. The input module 1210 may utilize a single antenna or a set of multiple antennas.

The output module 1215 may provide a means for transmitting signals generated by other components of the device 1205. For example, the output module 1215 may transmit information such as packets, user data, control information, or any combination thereof associated with various information channels (e.g., control channels, data channels, information channels related to illness detection techniques). In some examples, the output module 1215 may be co-located with the input module 1210 in a transceiver module. The output module 1215 may utilize a single antenna or a set of multiple antennas.

For example, the wearable application 1220 may include a data acquisition component 1225, an activity guidance component 1230, an operational mode component 1235, or any combination thereof. In some examples, the wearable application 1220, or various components thereof, may be configured to perform various operations (e.g., receiving, monitoring, transmitting) using or otherwise in cooperation with the input module 1210, the output module 1215, or both. For example, the wearable application 1220 may receive information from the input module 1210, send information to the output module 1215, or be integrated in combination with the input module 1210, the output module 1215, or both to receive information, transmit information, or perform various other operations as described herein.

The data acquisition component 1225 may be configured as or otherwise support a means for receiving physiological data associated with a user from a wearable device. The activity guidance component 1230 may be configured as or otherwise support a means for providing, to a user device associated with the user, a first set of physical activity targets and a first set of activity messages based at least in part on the received physiological data, the first set of physical activity targets and the first set of activity messages associated with a first operational mode associated with the user. The operational mode component 1235 may be configured as or otherwise support a means for identifying a trigger to transition from the first operational mode to a second operational mode associated with the user. The activity guidance component 1230 may be configured as or otherwise support a means for providing, to the user device based at least in part on identifying the trigger, a second set of physical activity targets and a second set of activity messages based at least in part on the received physiological data, the second set of physical activity targets and the second set of activity messages associated with the second operational mode.

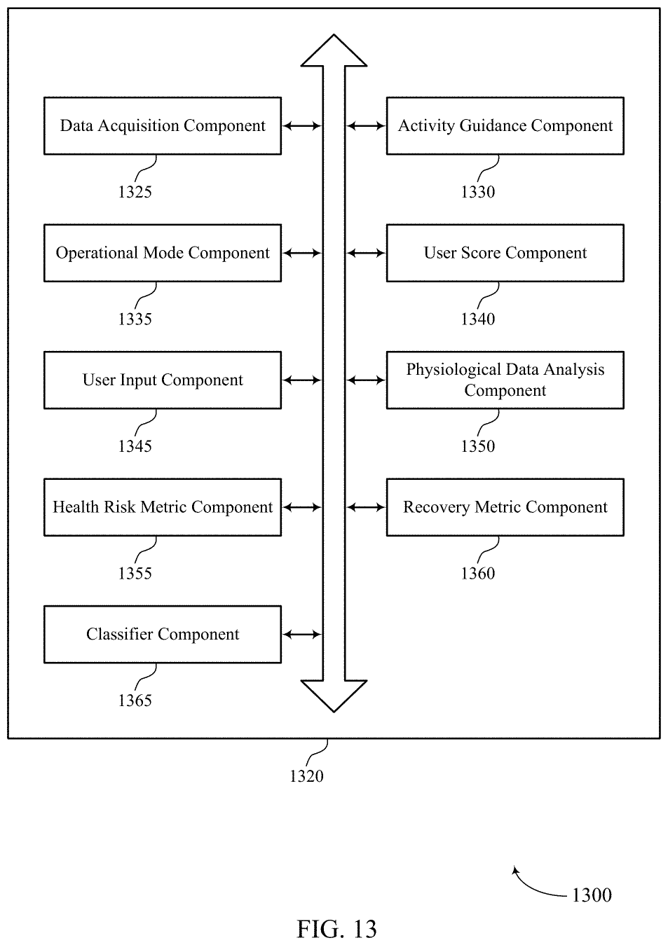

FIG. 13 shows a block diagram 1300 of a wearable application 1320 that supports techniques for providing guidance during rest and recovery in accordance with aspects of the present disclosure. The wearable application 1320 may be an example of aspects of a wearable application or a wearable application 1220, or both, as described herein. The wearable application 1320, or various components thereof, may be an example of means for performing various aspects of providing guidance during rest and recovery as described herein. For example, the wearable application 1320 may include a data acquisition component 1325, an activity guidance component 1330, an operational mode component 1335, a user score component 1340, a user input component 1345, a physiological data analysis component 1350, a health risk metric component 1355, a recovery metric component 1360, a classifier component 1365, or any combination thereof. Each of these components may communicate, directly or indirectly, with one another (e.g., via one or more buses).

The data acquisition component 1325 may be configured as or otherwise support a means for receiving physiological data associated with a user from a wearable device. The activity guidance component 1330 may be configured as or otherwise support a means for providing, to a user device associated with the user, a first set of physical activity targets and a first set of activity messages based at least in part on the received physiological data, the first set of physical activity targets and the first set of activity messages associated with a first operational mode associated with the user. The operational mode component 1335 may be configured as or otherwise support a means for identifying a trigger to transition from the first operational mode to a second operational mode associated with the user. In some examples, the activity guidance component 1330 may be configured as or otherwise support a means for providing, to the user device based at least in part on identifying the trigger, a second set of physical activity targets and a second set of activity messages based at least in part on the received physiological data, the second set of physical activity targets and the second set of activity messages associated with the second operational mode.

In some examples, the user score component 1340 may be configured as or otherwise support a means for determining, during a first time interval corresponding to the first operational mode, one or more scores associated with the user using a first algorithm and based at least in part on the received physiological data. In some examples, the user score component 1340 may be configured as or otherwise support a means for determining, during a second time interval corresponding to the second operational mode, the one or more scores associated with the user using a second algorithm different from the first algorithm and based at least in part on the received physiological data.

In some examples, the one or more scores comprise a Sleep Score, a Readiness Score, an Activity Score, or any combination thereof.

In some examples, the operational mode component 1335 may be configured as or otherwise support a means for identifying a second trigger to transition away from the second operational mode. In some examples, the operational mode component 1335 may be configured as or otherwise support a means for transitioning from the second operational mode to the first operational mode based at least in part on the second trigger. In some examples, the activity guidance component 1330 may be configured as or otherwise support a means for providing, to the user device based at least in part on transitioning to the first operational mode, the first set of physical activity targets and the first set of activity messages based at least in part on the received physiological data.

In some examples, the operational mode component 1335 may be configured as or otherwise support a means for identifying a second trigger to transition away from the second operational mode. In some examples, the operational mode component 1335 may be configured as or otherwise support a means for transitioning from the second operational mode to a third operational mode associated with the user based at least in part on the second trigger, wherein the third operational mode comprises an intermediary mode for transitioning from the second operational mode to the first operational mode. In some examples, the activity guidance component 1330 may be configured as or otherwise support a means for providing, to the user device based at least in part on transitioning to the third operational mode, a third set of physical activity targets and a third set of activity messages based at least in part on the received physiological data, the third set of physical activity targets and the third set of activity messages associated with the third operational mode.

In some examples, to support identifying the second trigger, the recovery metric component 1360 may be configured as or otherwise support a means for identifying that a recovery metric associated with the user satisfies a threshold recovery level for a period of time.

In some examples, the operational mode component 1335 may be configured as or otherwise support a means for identifying a third trigger to transition from the third operational mode to the first operational mode. In some examples, the operational mode component 1335 may be configured as or otherwise support a means for transitioning from the third operational mode to the first operational mode based at least in part on the third trigger. In some examples, the activity guidance component 1330 may be configured as or otherwise support a means for providing, to the user device based at least in part on transitioning to the first operational mode, the first set of physical activity targets and the first set of activity messages based at least in part on the received physiological data.

In some examples, identifying the third trigger is based at least in part on a duration of time spent in the second operational mode, measured physiological parameters included within the received physiological data that indicate the user has recovered to a sufficiently healthy level, or both.

In some examples, the first operational mode comprises a normal mode. In some examples, the second operational mode comprises a rest mode. In some examples, the third operational mode comprises a recovery mode.

In some examples, the user input component 1345 may be configured as or otherwise support a means for receiving, via the user device, a user input comprising an indication to transition from the first operational mode to the second operational mode, wherein identifying the trigger is based at least in part on receiving the user input.

In some examples, the physiological data analysis component 1350 may be configured as or otherwise support a means for identifying that the temperature data satisfies a temperature threshold, wherein identifying the trigger is based at least in part on the temperature data satisfying the temperature threshold.

In some examples, the health risk metric component 1355 may be configured as or otherwise support a means for identifying one or more health risk metrics associated with the user based at least in part on the received physiological data. In some examples, the health risk metric component 1355 may be configured as or otherwise support a means for identifying a potential health risk for the user based at least in part on the one or more health risk metrics associated with the user satisfying one or more thresholds, wherein identifying the trigger is based at least in part on identifying the potential health risk.

In some examples, the health risk metric component 1355 may be configured as or otherwise support a means for identifying the one or more health risk metrics associated with the user based at least in part on a plurality of physiological parameters associated with the physiological data, the one or more physiological parameters comprising temperature data, heart rate data, HRV data, respiratory rate data, blood oxygen saturation data, motion data, or any combination thereof.

In some examples, the health risk metric component 1355 may be configured as or otherwise support a means for identifying the one or more health risk metrics associated with the user based at least in part on one or more scores associated with the user, wherein the one or more scores comprise a Sleep Score, a Readiness Score, an Activity Score, or any combination thereof.

In some examples, the classifier component 1365 may be configured as or otherwise support a means for inputting the received physiological data into a classifier, wherein identifying the one or more health risk metrics is based at least in part on inputting the received physiological data into the classifier.

In some examples, to support identifying the trigger, the health risk metric component 1355 may be configured as or otherwise support a means for identifying a health risk metric associated with the user based at least in part on the received physiological data, the health risk metric associated with a relative probability that the user will transition from a healthy state to an unhealthy state. In some examples, to support identifying the trigger, the health risk metric component 1355 may be configured as or otherwise support a means for identifying that the health risk metric satisfies a health risk threshold.

In some examples, the activity guidance component 1330 may be configured as or otherwise support a means for selecting the second set of physical activity targets and the second set of activity messages based at least in part on the cause for transitioning from the first operational mode to the second operational mode.

In some examples, the first operational mode comprises a normal mode and the second operational mode comprises a rest mode. In some examples, the first set of physical activity targets comprise activity targets associated with the user when the user is in a healthy state. In some examples, the second set of physical activity targets comprise a set of reduced activity targets associated with the user when the user is in an unhealthy state or vulnerable state. In some examples, the second set of activity messages are configured to promote the set of reduced activity targets.

In some examples, the set of reduced activity targets are configured to promote recovery for the user.

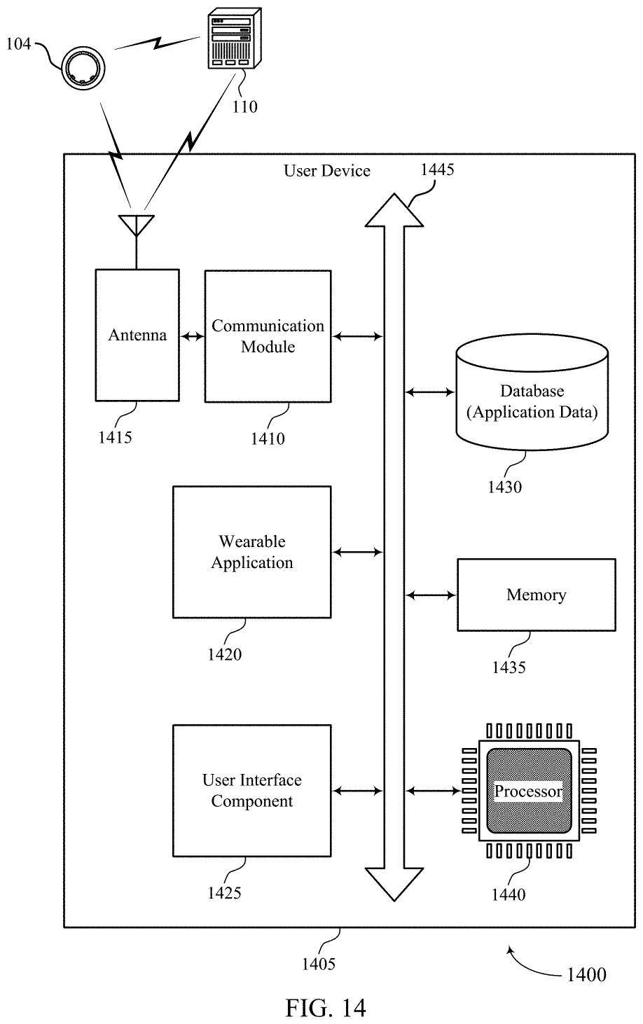

FIG. 14 shows a diagram of a system 1400 including a device 1405 that supports techniques for providing guidance during rest and recovery in accordance with aspects of the present disclosure. The device 1405 may be an example of or include the components of a device 1205 as described herein. The device 1405 may include an example of a user device 106, as described previously herein. The device 1405 may include components for bi-directional communications including components for transmitting and receiving communications with a wearable device 104 and a server 110, such as a wearable application 1420, a communication module 1410, an antenna 1415, a user interface component 1425, a database (application data) 1430, a memory 1435, and a processor 1440. These components may be in electronic communication or otherwise coupled (e.g., operatively, communicatively, functionally, electronically, electrically) via one or more buses (e.g., a bus 1445).

The communication module 1410 may manage input and output signals for the device 1405 via the antenna 1415. The communication module 1410 may include an example of the communication module 220-b of the user device 106 shown and described in FIG. 2. In this regard, the communication module 1410 may manage communications with the ring 104 and the server 110, as illustrated in FIG. 2. The communication module 1410 may also manage peripherals not integrated into the device 1405. In some cases, the communication module 1410 may represent a physical connection or port to an external peripheral. In some cases, the communication module 1410 may utilize an operating system such as iOS®, ANDROID®, MS-DOS®, MS-WINDOWS®, OS/2®, UNIX®, LINUX®, or another known operating system. In other cases, the communication module 1410 may represent or interact with a wearable device (e.g., ring 104), modem, a keyboard, a mouse, a touchscreen, or a similar device. In some cases, the communication module 1410 may be implemented as part of the processor 1440. In some examples, a user may interact with the device 1405 via the communication module 1410, user interface component 1425, or via hardware components controlled by the communication module 1410.

In some cases, the device 1405 may include a single antenna 1415. However, in some other cases, the device 1405 may have more than one antenna 1415, which may be capable of concurrently transmitting or receiving multiple wireless transmissions. The communication module 1410 may communicate bi-directionally, via the one or more antennas 1415, wired, or wireless links as described herein. For example, the communication module 1410 may represent a wireless transceiver and may communicate bi-directionally with another wireless transceiver. The communication module 1410 may also include a modem to modulate the packets, to provide the modulated packets to one or more antennas 1415 for transmission, and to demodulate packets received from the one or more antennas 1415.

The user interface component 1425 may manage data storage and processing in a database 1430. In some cases, a user may interact with the user interface component 1425. In other cases, the user interface component 1425 may operate automatically without user interaction. The database 1430 may be an example of a single database, a distributed database, multiple distributed databases, a data store, a data lake, or an emergency backup database.

The memory 1435 may include RAM and ROM. The memory 1435 may store computer-readable, computer-executable software including instructions that, when executed, cause the processor 1440 to perform various functions described herein. In some cases, the memory 1435 may contain, among other things, a basic I/O system (BIOS) which may control basic hardware or software operation such as the interaction with peripheral components or devices.

The processor 1440 may include an intelligent hardware device, (e.g., a general-purpose processor, a digital signal processor (DSP), a central processing unit (CPU), a microcontroller, an application-specific integrated circuit (ASIC), a field-programmable gate array (FPGA), a programmable logic device, a discrete gate or transistor logic component, a discrete hardware component, or any combination thereof). In some cases, the processor 1440 may be configured to operate a memory array using a memory controller. In other cases, a memory controller may be integrated into the processor 1440. The processor 1440 may be configured to execute computer-readable instructions stored in a memory 1435 to perform various functions (e.g., functions or tasks supporting a method and system for sleep staging algorithms).

For example, the wearable application 1420 may be configured as or otherwise support a means for receiving physiological data associated with a user from a wearable device. The wearable application 1420 may be configured as or otherwise support a means for providing, to a user device associated with the user, a first set of physical activity targets and a first set of activity messages based at least in part on the received physiological data, the first set of physical activity targets and the first set of activity messages associated with a first operational mode associated with the user. The wearable application 1420 may be configured as or otherwise support a means for identifying a trigger to transition from the first operational mode to a second operational mode associated with the user. The wearable application 1420 may be configured as or otherwise support a means for providing, to the user device based at least in part on identifying the trigger, a second set of physical activity targets and a second set of activity messages based at least in part on the received physiological data, the second set of physical activity targets and the second set of activity messages associated with the second operational mode.

The wearable application 1420 may include an application (e.g., “app”), program, software, or other component which is configured to facilitate communications with a ring 104, server 110, other user devices 106, and the like. For example, the wearable application 1420 may include an application executable on a user device 106 which is configured to receive data (e.g., physiological data) from a ring 104, perform processing operations on the received data, transmit and receive data with the servers 110, and cause presentation of data to a user 102.

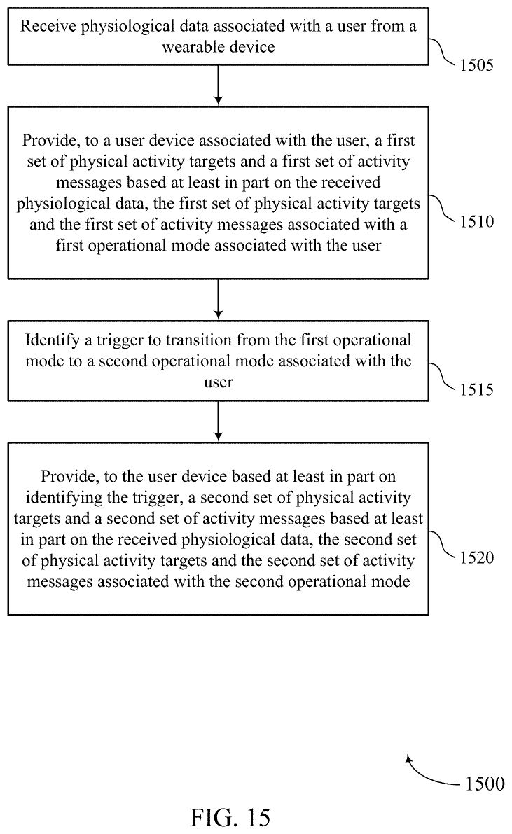

FIG. 15 shows a flowchart illustrating a method 1500 that supports techniques for providing guidance during rest and recovery in accordance with aspects of the present disclosure. The operations of the method 1500 may be implemented by a user device or its components as described herein. For example, the operations of the method 1500 may be performed by a user device as described with reference to FIGS. 1 through 14. In some examples, a user device may execute a set of instructions to control the functional elements of the user device to perform the described functions. Additionally or alternatively, the user device may perform aspects of the described functions using special-purpose hardware.

At 1505, the method may include receiving physiological data associated with a user from a wearable device. The operations of 1505 may be performed in accordance with examples as disclosed herein. In some examples, aspects of the operations of 1505 may be performed by a data acquisition component 1325 as described with reference to FIG. 13.

At 1510, the method may include providing, to a user device associated with the user, a first set of physical activity targets and a first set of activity messages based at least in part on the received physiological data, the first set of physical activity targets and the first set of activity messages associated with a first operational mode associated with the user. The operations of 1510 may be performed in accordance with examples as disclosed herein. In some examples, aspects of the operations of 1510 may be performed by an activity guidance component 1330 as described with reference to FIG. 13.

At 1515, the method may include identifying a trigger to transition from the first operational mode to a second operational mode associated with the user. The operations of 1515 may be performed in accordance with examples as disclosed herein. In some examples, aspects of the operations of 1515 may be performed by an operational mode component 1335 as described with reference to FIG. 13.

At 1520, the method may include providing, to the user device based at least in part on identifying the trigger, a second set of physical activity targets and a second set of activity messages based at least in part on the received physiological data, the second set of physical activity targets and the second set of activity messages associated with the second operational mode. The operations of 1520 may be performed in accordance with examples as disclosed herein. In some examples, aspects of the operations of 1520 may be performed by an activity guidance component 1330 as described with reference to FIG. 13.

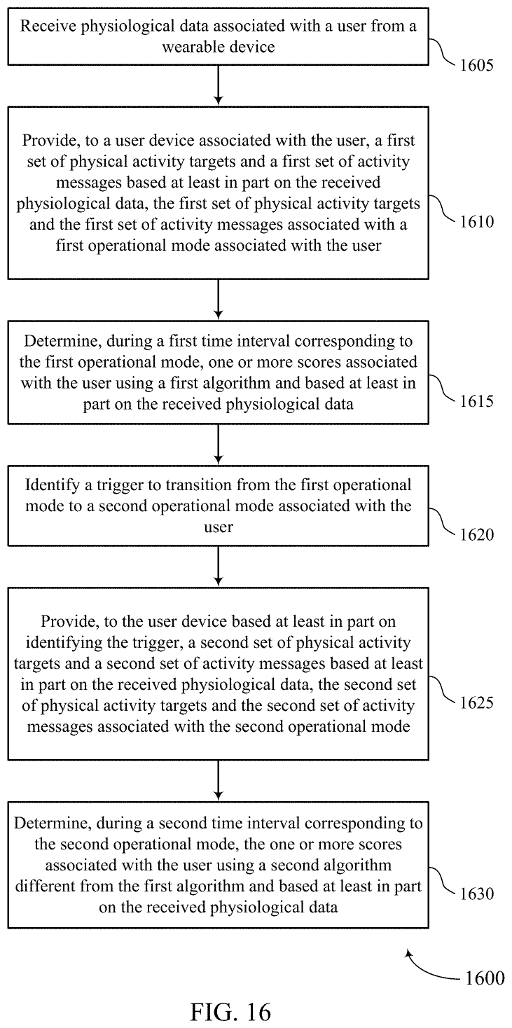

FIG. 16 shows a flowchart illustrating a method 1600 that supports techniques for providing guidance during rest and recovery in accordance with aspects of the present disclosure. The operations of the method 1600 may be implemented by a user device or its components as described herein. For example, the operations of the method 1600 may be performed by a user device as described with reference to FIGS. 1 through 14. In some examples, a user device may execute a set of instructions to control the functional elements of the user device to perform the described functions. Additionally or alternatively, the user device may perform aspects of the described functions using special-purpose hardware.

At 1605, the method may include receiving physiological data associated with a user from a wearable device. The operations of 1605 may be performed in accordance with examples as disclosed herein. In some examples, aspects of the operations of 1605 may be performed by a data acquisition component 1325 as described with reference to FIG. 13.

At 1610, the method may include providing, to a user device associated with the user, a first set of physical activity targets and a first set of activity messages based at least in part on the received physiological data, the first set of physical activity targets and the first set of activity messages associated with a first operational mode associated with the user. The operations of 1610 may be performed in accordance with examples as disclosed herein. In some examples, aspects of the operations of 1610 may be performed by an activity guidance component 1330 as described with reference to FIG. 13.

At 1615, the method may include determining, during a first time interval corresponding to the first operational mode, one or more scores associated with the user using a first algorithm and based at least in part on the received physiological data. The operations of 1615 may be performed in accordance with examples as disclosed herein. In some examples, aspects of the operations of 1615 may be performed by a user score component 1340 as described with reference to FIG. 13.

At 1620, the method may include identifying a trigger to transition from the first operational mode to a second operational mode associated with the user. The operations of 1620 may be performed in accordance with examples as disclosed herein. In some examples, aspects of the operations of 1620 may be performed by an operational mode component 1335 as described with reference to FIG. 13.

At 1625, the method may include providing, to the user device based at least in part on identifying the trigger, a second set of physical activity targets and a second set of activity messages based at least in part on the received physiological data, the second set of physical activity targets and the second set of activity messages associated with the second operational mode. The operations of 1625 may be performed in accordance with examples as disclosed herein. In some examples, aspects of the operations of 1625 may be performed by an activity guidance component 1330 as described with reference to FIG. 13.

At 1630, the method may include determining, during a second time interval corresponding to the second operational mode, the one or more scores associated with the user using a second algorithm different from the first algorithm and based at least in part on the received physiological data. The operations of 1630 may be performed in accordance with examples as disclosed herein. In some examples, aspects of the operations of 1630 may be performed by a user score component 1340 as described with reference to FIG. 13.

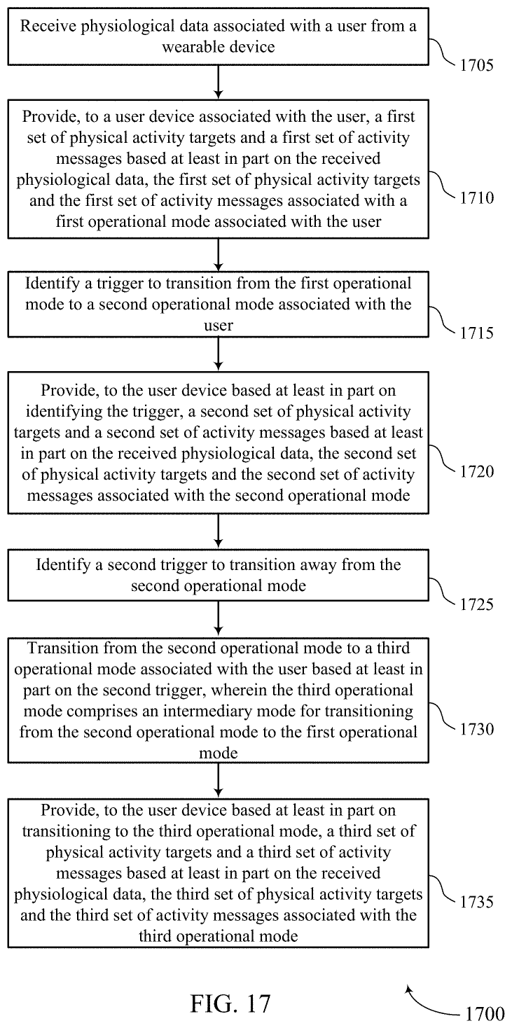

FIG. 17 shows a flowchart illustrating a method 1700 that supports techniques for providing guidance during rest and recovery in accordance with aspects of the present disclosure. The operations of the method 1700 may be implemented by a user device or its components as described herein. For example, the operations of the method 1700 may be performed by a user device as described with reference to FIGS. 1 through 14. In some examples, a user device may execute a set of instructions to control the functional elements of the user device to perform the described functions. Additionally or alternatively, the user device may perform aspects of the described functions using special-purpose hardware.

At 1705, the method may include receiving physiological data associated with a user from a wearable device. The operations of 1705 may be performed in accordance with examples as disclosed herein. In some examples, aspects of the operations of 1705 may be performed by a data acquisition component 1325 as described with reference to FIG. 13.

At 1710, the method may include providing, to a user device associated with the user, a first set of physical activity targets and a first set of activity messages based at least in part on the received physiological data, the first set of physical activity targets and the first set of activity messages associated with a first operational mode associated with the user. The operations of 1710 may be performed in accordance with examples as disclosed herein. In some examples, aspects of the operations of 1710 may be performed by an activity guidance component 1330 as described with reference to FIG. 13.

At 1715, the method may include identifying a trigger to transition from the first operational mode to a second operational mode associated with the user. The operations of 1715 may be performed in accordance with examples as disclosed herein. In some examples, aspects of the operations of 1715 may be performed by an operational mode component 1335 as described with reference to FIG. 13.

At 1720, the method may include providing, to the user device based at least in part on identifying the trigger, a second set of physical activity targets and a second set of activity messages based at least in part on the received physiological data, the second set of physical activity targets and the second set of activity messages associated with the second operational mode. The operations of 1720 may be performed in accordance with examples as disclosed herein. In some examples, aspects of the operations of 1720 may be performed by an activity guidance component 1330 as described with reference to FIG. 13.

At 1725, the method may include identifying a second trigger to transition away from the second operational mode. The operations of 1725 may be performed in accordance with examples as disclosed herein. In some examples, aspects of the operations of 1725 may be performed by an operational mode component 1335 as described with reference to FIG. 13.

At 1730, the method may include transitioning from the second operational mode to a third operational mode associated with the user based at least in part on the second trigger, wherein the third operational mode comprises an intermediary mode for transitioning from the second operational mode to the first operational mode. The operations of 1730 may be performed in accordance with examples as disclosed herein. In some examples, aspects of the operations of 1730 may be performed by an operational mode component 1335 as described with reference to FIG. 13.

At 1735, the method may include providing, to the user device based at least in part on transitioning to the third operational mode, a third set of physical activity targets and a third set of activity messages based at least in part on the received physiological data, the third set of physical activity targets and the third set of activity messages associated with the third operational mode. The operations of 1735 may be performed in accordance with examples as disclosed herein. In some examples, aspects of the operations of 1735 may be performed by an activity guidance component 1330 as described with reference to FIG. 13.

It should be noted that the methods described above describe possible implementations, and that the operations and the steps may be rearranged or otherwise modified and that other implementations are possible. Furthermore, aspects from two or more of the methods may be combined.

A method is described. The method may include receiving physiological data associated with a user from a wearable device, providing, to a user device associated with the user, a first set of physical activity targets and a first set of activity messages based at least in part on the received physiological data, the first set of physical activity targets and the first set of activity messages associated with a first operational mode associated with the user, identifying a trigger to transition from the first operational mode to a second operational mode associated with the user, and providing, to the user device based at least in part on identifying the trigger, a second set of physical activity targets and a second set of activity messages based at least in part on the received physiological data, the second set of physical activity targets and the second set of activity messages associated with the second operational mode.

An apparatus is described. The apparatus may include a processor, memory coupled with the processor, and instructions stored in the memory. The instructions may be executable by the processor to cause the apparatus to receive physiological data associated with a user from a wearable device, provide, to a user device associated with the user, a first set of physical activity targets and a first set of activity messages based at least in part on the received physiological data, the first set of physical activity targets and the first set of activity messages associated with a first operational mode associated with the user, identify a trigger to transition from the first operational mode to a second operational mode associated with the user, and provide, to the user device based at least in part on identifying the trigger, a second set of physical activity targets and a second set of activity messages based at least in part on the received physiological data, the second set of physical activity targets and the second set of activity messages associated with the second operational mode.

Another apparatus is described. The apparatus may include means for receiving physiological data associated with a user from a wearable device, means for providing, to a user device associated with the user, a first set of physical activity targets and a first set of activity messages based at least in part on the received physiological data, the first set of physical activity targets and the first set of activity messages associated with a first operational mode associated with the user, means for identifying a trigger to transition from the first operational mode to a second operational mode associated with the user, and means for providing, to the user device based at least in part on identifying the trigger, a second set of physical activity targets and a second set of activity messages based at least in part on the received physiological data, the second set of physical activity targets and the second set of activity messages associated with the second operational mode.

A non-transitory computer-readable medium storing code is described. The code may include instructions executable by a processor to receive physiological data associated with a user from a wearable device, provide, to a user device associated with the user, a first set of physical activity targets and a first set of activity messages based at least in part on the received physiological data, the first set of physical activity targets and the first set of activity messages associated with a first operational mode associated with the user, identify a trigger to transition from the first operational mode to a second operational mode associated with the user, and provide, to the user device based at least in part on identifying the trigger, a second set of physical activity targets and a second set of activity messages based at least in part on the received physiological data, the second set of physical activity targets and the second set of activity messages associated with the second operational mode.

Some examples of the method, apparatuses, and non-transitory computer-readable medium described herein may further include operations, features, means, or instructions for determining, during a first time interval corresponding to the first operational mode, one or more scores associated with the user using a first algorithm and based at least in part on the received physiological data and determining, during a second time interval corresponding to the second operational mode, the one or more scores associated with the user using a second algorithm different from the first algorithm and based at least in part on the received physiological data.

In some examples of the method, apparatuses, and non-transitory computer-readable medium described herein, the one or more scores comprise a Sleep Score, a Readiness Score, an Activity Score, or any combination thereof.

Some examples of the method, apparatuses, and non-transitory computer-readable medium described herein may further include operations, features, means, or instructions for identifying a second trigger to transition away from the second operational mode, transitioning from the second operational mode to the first operational mode based at least in part on the second trigger, and providing, to the user device based at least in part on transitioning to the first operational mode, the first set of physical activity targets and the first set of activity messages based at least in part on the received physiological data.

Some examples of the method, apparatuses, and non-transitory computer-readable medium described herein may further include operations, features, means, or instructions for identifying a second trigger to transition away from the second operational mode, transitioning from the second operational mode to a third operational mode associated with the user based at least in part on the second trigger, wherein the third operational mode comprises an intermediary mode for transitioning from the second operational mode to the first operational mode, and providing, to the user device based at least in part on transitioning to the third operational mode, a third set of physical activity targets and a third set of activity messages based at least in part on the received physiological data, the third set of physical activity targets and the third set of activity messages associated with the third operational mode.

In some examples of the method, apparatuses, and non-transitory computer-readable medium described herein, identifying the second trigger may include operations, features, means, or instructions for identifying that a recovery metric associated with the user satisfies a threshold recovery level for a period of time.

Some examples of the method, apparatuses, and non-transitory computer-readable medium described herein may further include operations, features, means, or instructions for identifying a third trigger to transition from the third operational mode to the first operational mode, transitioning from the third operational mode to the first operational mode based at least in part on the third trigger, and providing, to the user device based at least in part on transitioning to the first operational mode, the first set of physical activity targets and the first set of activity messages based at least in part on the received physiological data.

Some examples of the method, apparatuses, and non-transitory computer-readable medium described herein may further include operations, features, means, or instructions for identifying the third trigger may be based at least in part on a duration of time spent in the second operational mode, measured physiological parameters included within the received physiological data that indicate the user may have recovered to a sufficiently healthy level, or both.

In some examples of the method, apparatuses, and non-transitory computer-readable medium described herein, the first operational mode comprises a normal mode, the second operational mode comprises a rest mode, and the third operational mode comprises a recovery mode.

Some examples of the method, apparatuses, and non-transitory computer-readable medium described herein may further include operations, features, means, or instructions for receiving, via the user device, a user input comprising an indication to transition from the first operational mode to the second operational mode, wherein identifying the trigger may be based at least in part on receiving the user input.

In some examples of the method, apparatuses, and non-transitory computer-readable medium described herein, and the method, apparatuses, and non-transitory computer-readable medium may include further operations, features, means, or instructions for identifying that the temperature data satisfies a temperature threshold, wherein identifying the trigger may be based at least in part on the temperature data satisfying the temperature threshold.

Some examples of the method, apparatuses, and non-transitory computer-readable medium described herein may further include operations, features, means, or instructions for identifying one or more health risk metrics associated with the user based at least in part on the received physiological data and identifying a potential health risk for the user based at least in part on the one or more health risk metrics associated with the user satisfying one or more thresholds, wherein identifying the trigger may be based at least in part on identifying the potential health risk.

Some examples of the method, apparatuses, and non-transitory computer-readable medium described herein may further include operations, features, means, or instructions for identifying the one or more health risk metrics associated with the user based at least in part on a plurality of physiological parameters associated with the physiological data, the one or more physiological parameters comprising temperature data, heart rate data, HRV data, respiratory rate data, blood oxygen saturation data, motion data, or any combination thereof.

Some examples of the method, apparatuses, and non-transitory computer-readable medium described herein may further include operations, features, means, or instructions for identifying the one or more health risk metrics associated with the user based at least in part on one or more scores associated with the user, wherein the one or more scores comprise a Sleep Score, a Readiness Score, an Activity Score, or any combination thereof.

Some examples of the method, apparatuses, and non-transitory computer-readable medium described herein may further include operations, features, means, or instructions for inputting the received physiological data into a classifier, wherein identifying the one or more health risk metrics may be based at least in part on inputting the received physiological data into the classifier.

In some examples of the method, apparatuses, and non-transitory computer-readable medium described herein, identifying the trigger may include operations, features, means, or instructions for identifying a health risk metric associated with the user based at least in part on the received physiological data, the health risk metric associated with a relative probability that the user will transition from a healthy state to an unhealthy state and identifying that the health risk metric satisfies a health risk threshold.

In some examples of the method, apparatuses, and non-transitory computer-readable medium described herein, and the method, apparatuses, and non-transitory computer-readable medium may include further operations, features, means, or instructions for selecting the second set of physical activity targets and the second set of activity messages based at least in part on the cause for transitioning from the first operational mode to the second operational mode.

In some examples of the method, apparatuses, and non-transitory computer-readable medium described herein, the first operational mode comprises a normal mode and the second operational mode comprises a rest mode, the first set of physical activity targets comprise activity targets associated with the user when the user may be in a healthy state, the second set of physical activity targets comprise a set of reduced activity targets associated with the user when the user may be in an unhealthy state or vulnerable state, and the second set of activity messages may be configured to promote the set of reduced activity targets.

In some examples of the method, apparatuses, and non-transitory computer-readable medium described herein, the set of reduced activity targets may be configured to promote recovery for the user.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method comprising:
receiving physiological data associated with a user from a wearable device;
providing, to a user device associated with the user, a first set of physical activity targets and a first set of activity messages based at least in part on the received physiological data, the first set of physical activity targets and the first set of activity messages associated with a first operational mode associated with the user;
identifying a trigger to transition from the first operational mode to a second operational mode associated with the user; and
providing, to the user device based at least in part on identifying the trigger, a second set of physical activity targets and a second set of activity messages based at least in part on the received physiological data, the second set of physical activity targets and the second set of activity messages associated with the second operational mode.

2. The method of claim 1, further comprising:
determining, during a first time interval corresponding to the first operational mode, one or more scores associated with the user using a first algorithm and based at least in part on the received physiological data; and
determining, during a second time interval corresponding to the second operational mode, the one or more scores associated with the user using a second algorithm different from the first algorithm and based at least in part on the received physiological data.

3. The method of claim 2, wherein the one or more scores comprise a sleep score, a readiness score, an activity score, or any combination thereof.

4. The method of claim 1, further comprising:
identifying a second trigger to transition away from the second operational mode;
transitioning from the second operational mode to the first operational mode based at least in part on the second trigger; and
providing, to the user device based at least in part on transitioning to the first operational mode, the first set of physical activity targets and the first set of activity messages based at least in part on the received physiological data.

5. The method of claim 1, further comprising:
identifying a second trigger to transition away from the second operational mode;
transitioning from the second operational mode to a third operational mode associated with the user based at least in part on the second trigger, wherein the third operational mode comprises an intermediary mode for transitioning from the second operational mode to the first operational mode; and
providing, to the user device based at least in part on transitioning to the third operational mode, a third set of physical activity targets and a third set of activity messages based at least in part on the received physiological data, the third set of physical activity targets and the third set of activity messages associated with the third operational mode.

6. The method of claim 5, wherein identifying the second trigger comprises:
identifying that a recovery metric associated with the user satisfies a threshold recovery level for a period of time.

7. The method of claim 5, further comprising:
identifying a third trigger to transition from the third operational mode to the first operational mode;
transitioning from the third operational mode to the first operational mode based at least in part on the third trigger; and
providing, to the user device based at least in part on transitioning to the first operational mode, the first set of physical activity targets and the first set of activity messages based at least in part on the received physiological data.

8. The method of claim 7, wherein identifying the third trigger is based at least in part on a duration of time spent in the second operational mode, measured physiological parameters included within the received physiological data that indicate the user has recovered to a sufficiently healthy level, or both.

9. The method of claim 5, wherein the first operational mode comprises a normal mode, wherein the second operational mode comprises a rest mode, and wherein the third operational mode comprises a recovery mode.

10. The method of claim 1, further comprising:
receiving, via the user device, a user input comprising an indication to transition from the first operational mode to the second operational mode, wherein identifying the trigger is based at least in part on receiving the user input.

11. The method of claim 1, wherein the physiological data comprises temperature data, the method further comprising:
identifying that the temperature data satisfies a temperature threshold, wherein identifying the trigger is based at least in part on the temperature data satisfying the temperature threshold.

12. The method of claim 1, further comprising:
identifying one or more health risk metrics associated with the user based at least in part on the received physiological data; and
identifying a potential health risk for the user based at least in part on the one or more health risk metrics associated with the user satisfying one or more thresholds, wherein identifying the trigger is based at least in part on identifying the potential health risk.

13. The method of claim 12, further comprising:
identifying the one or more health risk metrics associated with the user based at least in part on a plurality of physiological parameters associated with the physiological data, the one or more physiological parameters comprising temperature data, heart rate data, heart rate variability data, respiratory rate data, blood oxygen saturation data, motion data, or any combination thereof.

14. The method of claim 12, further comprising:
identifying the one or more health risk metrics associated with the user based at least in part on one or more scores associated with the user, wherein the one or more scores comprise a sleep score, a readiness score, an activity score, or any combination thereof.

15. The method of claim 12, further comprising:
inputting the received physiological data into a classifier, wherein identifying the one or more health risk metrics is based at least in part on inputting the received physiological data into the classifier.

16. The method of claim 1, wherein identifying the trigger comprises:
identifying a health risk metric associated with the user based at least in part on the received physiological data, the health risk metric associated with a relative probability that the user will transition from a healthy state to an unhealthy state; and
identifying that the health risk metric satisfies a health risk threshold.

17. The method of claim 1, wherein the trigger to transition from the first operational mode to the second operational mode comprises an indication of a cause for transitioning from the first operational mode to the second operational mode, the method further comprising:
selecting the second set of physical activity targets and the second set of activity messages based at least in part on the cause for transitioning from the first operational mode to the second operational mode.

18. The method of claim 1, wherein the first operational mode comprises a normal mode and the second operational mode comprises a rest mode, wherein the first set of physical activity targets comprise activity targets associated with the user when the user is in a healthy state, wherein the second set of physical activity targets comprise a set of reduced activity targets associated with the user when the user is in an unhealthy state or vulnerable state, and wherein the second set of activity messages are configured to promote the set of reduced activity targets.

19. The method of claim 18, wherein the set of reduced activity targets are configured to promote recovery for the user.

20. An apparatus, comprising:
a processor;
memory coupled with the processor; and
instructions stored in the memory and executable by the processor to cause the apparatus to:
receive physiological data associated with a user from a wearable device;
provide, to a user device associated with the user, a first set of physical activity targets and a first set of activity messages based at least in part on the received physiological data, the first set of physical activity targets and the first set of activity messages associated with a first operational mode associated with the user;
identify a trigger to transition from the first operational mode to a second operational mode associated with the user; and
provide, to the user device based at least in part on identifying the trigger, a second set of physical activity targets and a second set of activity messages based at least in part on the received physiological data, the second set of physical activity targets and the second set of activity messages associated with the second operational mode.
